(19) United States
(12) Patent Application Publication
(10) Pub. No.: US 2023/0372313 A1
(43) Pub. Date: Nov. 23, 2023

(54) METHODS FOR REDUCING GLYCOSPHINGOLIPID CONCENTRATION IN BRAIN TISSUE AND METHODS OF TREATMENT OF NEURODEGENERATIVE DISEASES INVOLVING THE SAME

(71) Applicant: GENZYME CORPORATION, Cambridge, MA (US)

(72) Inventors: Tanya Zaremba FISCHER, Newton, MA (US); Judith PETERSCHMITT, Belmont, MA (US)

(21) Appl. No.: 18/007,433

(22) PCT Filed: Jul. 30, 2021
(86) PCT No.: PCT/IB2021/056971

§ 371 Date: Jan. 30, 2023

Related U.S. Application Data
(60) Provisional application No. 63/059,047, filed on Jul. 30, 2020.
(60) Provisional application No. 63/131,120, filed on Dec. 28, 2020.

(51) Int. Cl. A61K 31/439; A61P 25/16

(52) U.S. Cl. CPC A61K 31/439; A61P 25/16

(57) ABSTRACT

Provided are methods for treating or preventing Parkinson's disease or dementia with Lewy Bodies in a human subject. The methods target lipid dysregulation in brain tissue of the subject and can, in particular, reduce the concentration of glycosphingolipids in brain tissue. The methods use quinuclidine compounds of formula (I), or pharmaceutically acceptable prodrugs or salts thereof. In particular, the human subject may be a carrier of one or more glucocerebrosidase 1 gene (GBA1) mutations, e.g. a heterozygous carrier of GBA1 mutations. Also provided are methods for assessing the effectiveness of treatments as described herein.

The present disclosure relates to improved methods for treating or preventing Parkinson's disease or dementia with Lewy Bodies in a human subject. The methods target lipid dysregulation in brain tissue of the subject and can, in particular, reduce the concentration of glycosphingolipids in brain tissue. The methods use quinuclidine compounds of formula (I), including (S)-quinuclidin-3-yl (2-(2-(4-fluorophenyl)thiazol-4-yl)propan-2-yl)carbamate, or pharmaceutically acceptable salts or prodrugs thereof. In particular, the human subject may be a carrier of one or more glucocerebrosidase 1 gene (GBA1) mutations, e.g. a heterozygous carrier of GBA1 mutations. This disclosure also relates to methods for assessing the effectiveness of treatments as described herein.

### BACKGROUND OF THE INVENTION

Mutations of the GBA1 gene, encoding the lysosomal enzyme glucocerebrosidase, increase the risk of developing Parkinson's disease (“PD”) in those who carry them (Migdalska-Richards et al., J Neurochem 2016; 139:77-90). This risk is increased 5-fold in heterozygous GBA1 mutation carriers, and 10-20 fold in homozygous carriers (Riboldi et al., Cells 2019; 8(4):364). Homozygous carriers of GBA1 mutations (loss-of-function) typically suffer from Gaucher disease, a lysosomal storage disorder. Although the majority of GBA1 mutation carriers will not develop synucleinopathies, an estimated 7%-10% of patients with PD carry a GBA1 mutation, with prevalence varying depending on ethnicity and geography (Blandini et al., Mov Disord 2019; 34: 9-21). These mutations are associated with earlier disease onset and more rapid cognitive decline in some patients. GBA1 mutations are also attributed to dementia with Lewy Bodies (DLB).

The exact mechanism by which GBA1 mutations contribute to PD or DLB pathogenesis is unclear. Studies on a mouse model of Gaucher-related synucleinopathy (Gba^{D409V/D409V}) which presents with progressive accumulation of α-synuclein aggregates in the CNS as well as memory deficits, have shown that treatment with a brain-penetrant GCS inhibitor can reduce hippocampal pathological α-synuclein aggregate accumulation and symptoms of synucleinopathy (Sardi et al., Proc Natl Acad Sci USA 2017; 114: 2699-704). However, the relevance of these results to lipid dysregulation in the brain tissue of human subjects, especially those who carry heterozygous GBA1 mutations, has not been determined. There remains, therefore, a need to elucidate the impact of modulating glycolipid levels in the CNS, and to develop improved methods for the treatment of PD and DLB in humans.

The present inventors have surprisingly discovered that compounds as described herein are capable of in vivo modulation of glycosphingolipid levels in the CNS of human subjects which do not have a lysosomal storage disease (such as Gaucher disease). This modulation of glycosphingolipid levels is reflected in a marked reduction in glucosylceramide (“GL-1”) concentration in biological fluids such as cerebrospinal fluid (“CSF”). Moreover, the reduction in GL-1 levels in the CSF also correlates in a dose-dependent manner with the concentration of the compound which is administered. Thus, the inventors have determined that modulation of the glycosphingolipid profile in brain tissue of a human subject with PD is achievable via oral administration of a compound as described herein. Without wishing to be bound by theory, the inventors postulate that the compounds of the invention are able to have a positive impact on glycosphingolipid dysregulation in the CNS. This can lead to improved lysosomal function, improved processing of the neuronal protein α-synuclein and a reduction in neuronal dysfunction, even in subjects carrying a heterozygous GBA1 mutation. This is of particular significance when patients with PD only have symptomatic treatments available (such as levodopa, dopamine agonists, and/or deep brain stimulation), and hence, there is currently an unmet medical need for the development of treatments that slow, stop, or even reverse, neurodegeneration.

### SUMMARY OF THE INVENTION

Accordingly, in a first aspect the present invention provides a method of treating or preventing a neurodegenerative disease in a human subject by reducing glycosphingolipid concentration in brain tissue of the subject, whereby the concentration of glucosylceramide (GL-1) in the cerebrospinal fluid (CSF) of the subject is reduced by at least 30%, wherein the neurodegenerative disease is selected from Parkinson's disease (PD) and dementia with Lewy Bodies (DLB), and wherein the subject does not have (e.g. has not been diagnosed as having, or being at risk of having) a lysosomal storage disease; the method comprising administering to the subject an effective amount of a compound of formula (I),

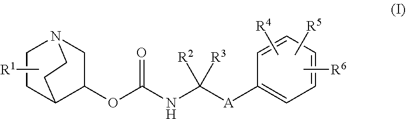

or a pharmaceutically acceptable salt or prodrug thereof, wherein:
R¹ is selected from hydrogen, halogen (e.g. fluorine), cyano, nitro, hydroxy, thio, amino, C₁₋₆-alkyl (e.g. methyl or ethyl), C₂₋₆-alkenyl, C₂₋₆-alkynyl, C₁₋₆-alkyloxy, C₂₋₆-alkenyloxy, and C₂₋₆-alkynyloxy, wherein said alkyl, alkenyl, alkynyl, alkyloxy, alkenyloxy, or alkynyloxy is optionally substituted with one or more (e.g. 1, 2, or 3) groups selected from halogen, cyano, nitro, hydroxy, thio, or amino;
R² and R³ are independently selected from C₁₋₃-alkyl, optionally substituted by one or more (e.g. 1, 2, or 3) halogens, or R² and R³ together form a cyclopropyl or cyclobutyl group, optionally substituted by one or more (e.g. 1 or 2) halogens;
R⁴, R⁵, and R⁶ are each independently selected from hydrogen, halogen, nitro, hydroxy, thio, amino, C₁₋₆-alkyl, and C₁₋₆-alkyloxy, wherein said alkyl or alkyloxy is optionally substituted by one or more (e.g. 1, 2, or 3) groups selected from halogen, hydroxy, cyano, and C₁₋₆-alkyloxy; and
A is a 5- or 6-membered aryl or heteroaryl group (e.g. phenyl or thiazolyl), optionally substituted with 1, 2, or 3 groups independently selected from halogen, hydroxy, thio, amino, nitro, C₁₋₆-alkyloxy, and C₁₋₆-alkyl.

In embodiments, R¹ is selected from hydrogen, fluorine, methyl, and ethyl, wherein said methyl or ethyl is optionally substituted by 1 or 2 groups selected from halogen, hydroxy, thio, or amino.

In embodiments R² and R³ are each independently selected from methyl and ethyl groups, optionally substituted with one or more fluorines.

In embodiments, R⁴ is selected from a halogen (e.g. fluorine), C₁₋₃-alkyl (e.g. methyl), and C₁₋₃-alkyloxy (e.g. methoxy or ethoxy), wherein said alkyl or alkyloxy is optionally substituted by one or more (e.g. 1, 2, or 3) groups selected from a halogen and C₁₋₃-alkyloxy (e.g. methoxy or ethoxy).

In embodiments, R⁵ and R⁶ are each hydrogen. In embodiments, R⁴ is fluorine or 2-methoxyethoxy, and R⁵ and R⁶ are hydrogen.

In embodiments, R⁴ is positioned at the 4-position of the phenyl ring to which it is attached (i.e. para to the A substituent).

In embodiments, A is phenyl, optionally substituted with 1, 2, or 3 groups independently selected from halogen, hydroxy, thio, amino, nitro, C₁₋₆alkyloxy, and C₁₋₆alkyl (e.g. methyl). In one embodiment, the two groups attached to the A substituent are positioned in a 1,3- or a 1,4-relationship to each other (i.e. meta or para). In other embodiments, A is a 5-membered heteroaryl group which contains 1 or 2 heteroatoms selected from N and S. In one embodiment, the two groups attached to the A substituent are positioned in a 1,3-relationship to each other (i.e. meta).

In embodiments, said compound is a compound of formula (II), (III), or (IV),

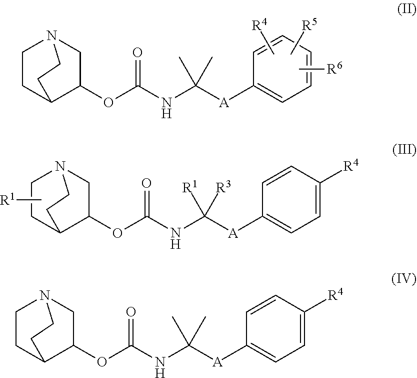

or a pharmaceutically acceptable salt or prodrug thereof.

In embodiments, said compound is a compound of formula (V),

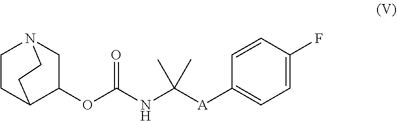

or a pharmaceutically acceptable salt or prodrug thereof.

In embodiments, said compound is a compound of formula (VI), (VII), or (VIII),

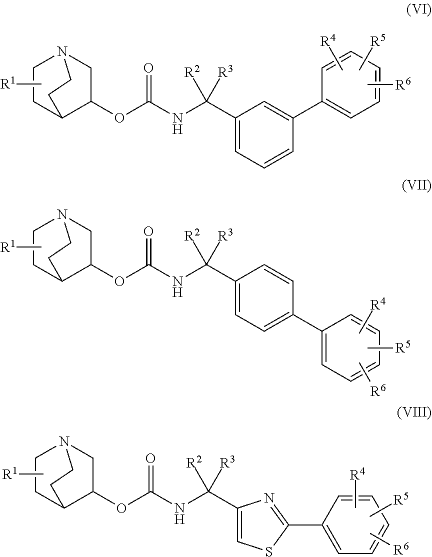

or a pharmaceutically acceptable salt or prodrug thereof.

In embodiments, said compound is a compound of formula (IX) or (XI),

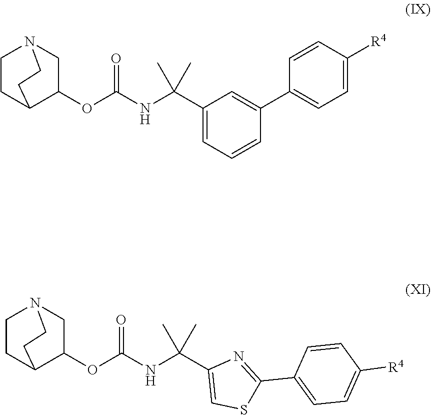

or a pharmaceutically acceptable salt or prodrug thereof. In one embodiment, R⁴ is fluorine.

In embodiments, said compound is selected from: quinuclidin-3-yl (2-(4′-fluoro-[1,1′-biphenyl]-3-yl)propan-2-yl)carbamate; (S)-quinuclidin-3-yl (2-(2-(4-fluorophenyl)thiazol-4-yl)propan-2-yl)carbamate; (S)-quinuclidin-3-yl (2-(4′-(2-methoxyethoxy)-[1,1′-biphenyl]-4-yl)propan-2-yl)carbamate; and the pharmaceutically acceptable salts and prodrugs thereof. In one embodiment, said compound is quinuclidin-3-yl (2-(4′-fluoro-[1,1′-biphenyl]-3-yl)propan-2-yl)carbamate. In another embodiment, said compound is (S)-quinuclidin-3-yl (2-(2-(4-fluorophenyl)thiazol-4-yl)propan-2-yl)carbamate. In one embodiment, the compound is an acid addition salt form of (S)-quinuclidin-3-yl (2-(2-(4-fluorophenyl)thiazol-4-yl)propan-2-yl)carbamate, selected from the hydrochloride, hydroxysuccinate, and malate.

In embodiments, the subject is a heterozygous carrier of one or more glucocerebrosidase 1 gene (GBA1) mutations. The one or more gene (GBA1) mutations may, for instance, be selected from severe mutations (e.g. those denoted as a “severe GBA mutation” in Table 1, such as L444P) and other (i.e. non-severe) GBA1 mutations (e.g. those denoted as an “other GBA mutation” in Table 1, such as N370S). In embodiments, the one or more GBA1 mutations are selected from L444P, 84GG, A456P, R120W, D409H, E235A, E340A, N370S, E326K, R496C, G193W, T369M, R496H, and S271G. For example, the subject is a carrier of a L444P mutation, and optionally one or more further GBA1 mutations. In another example, the subject is a carrier of a N370S mutation, and optionally one or more further GBA1 mutations.

In embodiments, the brain tissue is (or comprises) neurons.

In a further aspect, the invention provides a method of treating or preventing a neurodegenerative disease in a human subject by reducing glycosphingolipid concentration in brain tissue of the subject, whereby the concentration of glucosylceramide (GL-1) in the cerebrospinal fluid (CSF) of the subject is reduced by at least 30%, wherein the neurodegenerative disease is selected from Parkinson's disease (PD) and dementia with Lewy Bodies (DLB), and wherein the subject is a heterozygous carrier of one or more glucocerebrosidase 1 gene (GBA1) mutations; the method comprising administering to the subject an effective amount of a compound of formula (I) as defined hereinbefore.

In embodiments, the one or more GBA1 mutations are selected from L444P, 84GG, A456P, R120W, D409H, E235A, E340A, N370S, E326K, R496C, G193W, T369M, R496H, and S271G. For example, the subject is a carrier of a L444P mutation, and optionally one or more further GBA1 mutations. In another example, the subject is a carrier of a N370S mutation, and optionally one or more further GBA1 mutations.

In embodiments, the method results in a reduction in GL-1 concentration in CSF of at least 40%, e.g. at least 50%, at least 60%, or at least 70%.

In embodiments, the method results in the reduction in GL-1 concentration in the CSF within 3 months of commencing treatment, e.g. within 2 months, 4 weeks, 2 weeks, or 1 week of commencing treatment.

In embodiments, said subject has been diagnosed as having Parkinson's disease. In other embodiments, said subject has been diagnosed as being at risk of developing Parkinson's disease, and the method prevents or delays the onset and/or development of Parkinson's disease in the subject.

In embodiments, the subject has a diagnosis of Parkinson's disease, and the method prevents, reduces, or reverses motor dysfunction (e.g. tremor), bradykinesia, rigidity, postural instability, and/or impaired balance. In one embodiment, the subject has a diagnosis of Parkinson's disease and the subject has at least one of the following characteristics: i) a family history of Parkinson's disease; ii) a baseline Montreal Cognitive Assessment (MoCA) score of ≤26 (for example, from 20 to 25); and iii) a Movement Disorder Society-Unified Parkinson's Disease Rating Scale (MDS-UPDRS) (Part II+III) score of at least 35. In one embodiment, the subject has a diagnosis of early-stage Parkinson's disease characterized by: i) at least two of the following conditions: resting tremor, postural instability, akinesia/hypokinesia, and muscle rigidity; ii) a Hoehn and Yahr Scale stage of ≤2 at baseline; and/or iii) a Parkinson's diagnosis of ≥2 years.

In embodiments, the subject has been treated historically with medications selected from dihydroxyphenylalanine (DOPA) or derivatives thereof (e.g. levodopa/carbidopa), monoamine oxidase B inhibitors (e.g. rasagiline or selegiline), dopamine agonists (e.g. ropinirole, bromocriptine, cabergoline, pergolide, pramipexole, or apomorphine), Catechol-O-methyltransferase inhibitors (e.g. entacapone, tolcapone), anticholinergics (e.g. artane, cogentin), adamantane derivatives, and/or acetylcholinesterase inhibitors (e.g. tacrine, rivastigmine, galantamine, donepezil, or memantine).

In embodiments, the subject has a diagnosis of dementia with Lewy Bodies (DLB). In one embodiment, said method prevents, reduces, or reverses the progression of dementia in the subject.

In a further aspect, the invention provides a method of reducing glycosphingolipid concentration in brain tissue of a human subject in need thereof, whereby the concentration of glucosylceramide (GL-1) in the cerebrospinal fluid (CSF) of the subject is reduced as a result of the method, wherein the subject does not have (e.g. has not been diagnosed as having, or being at risk of having) a lysosomal storage disease; the method comprising administering to the subject an effective amount of a compound of formula (I) as defined hereinbefore.

In embodiments, the method results in a reduction in GL-1 concentration in CSF of at least 30%, e.g. at least 40%, at least 50%, at least 60%, or at least 70%.

In embodiments, the method results in the reduction in GL-1 concentration in the CSF is within 3 months of commencing treatment, e.g. within 2 months, 1 month, 3 weeks, 2 weeks, or 1 week of commencing treatment.

In embodiments, said brain tissue is a neuron of the substantia nigra, cerebral cortex, hippocampus, frontal lobes, and/or temporal lobes of said subject.

In embodiments, said compound, or pharmaceutically acceptable salt or prodrug thereof, is administered by systemic administration, e.g. via a non-parenteral route. In one embodiment, said compound, or pharmaceutically acceptable salt or prodrug thereof, is administered orally.

In embodiments, said subject is administered a daily dose of about 2 mg to about 30 mg of said compound, or pharmaceutically acceptable salt or prodrug thereof, e.g. from 2 mg to 20 mg, or from 2 mg to 10 mg, or from 4 mg to 8 mg, or from 10 to 20 mg, or from 4 mg to 15 mg, or a dose selected from 4 mg, 8 mg, and 15 mg.

In embodiments, the method prevents, reduces, or reverses deterioration in cognitive domains in the subject.

In embodiments, the method is effective to improve cognitive ability or reduce cognitive deficits in the subject as measured by a reduction in the time taken to complete the trail-making test (TMT), TMT-A and/or TMT-B, a reduction in the difference between TMT-A time and TMT-B time (TMT-B−TMT-A), for example, a reduction of at least 10%, or at least 20%, or at least 30%, or at least 40%, or at least 50% (e.g. wherein TMT-A decreases by 5-20%, and/or TMT-B decreases by 25-30%, and/or [TMT-B−TMT-A] decreases by 25-30%). In one embodiment, the method prevents, reduces, or reverses deterioration in attention and concentration, executive functions, memory (e.g. working memory), language, visuo-constructional skills, conceptual thinking, calculations, orientation, decision making, and/or problem solving.

In embodiments, the subject is a heterozygous carrier of one or more glucocerebrosidase 1 gene (GBA1) mutations (e.g. L444P and/or N370S), and the compound is a malate acid addition salt of (S)-quinuclidin-3-yl (2-(2-(4-fluorophenyl)thiazol-4-yl)propan-2-yl)carbamate and is orally administered at a daily dose of about 15 mg (measured as the equivalent amount of free base). In one embodiment, the concentration of the compound (measured as the equivalent amount of free base) in the CSF of the subject is at least 4 ng/ml, e.g. at least 8 ng/ml, or at least 10 ng/ml, within 3 months of commencing treatment, e.g. within 2 months, 1 month, 3 weeks, 2 weeks, or 1 week of commencing treatment.

In embodiments, the method results in increased blood flow in the brain (e.g. in one or more of the frontal, occipital, parietal, or temporal lobes), for example, as shown by fMRI imaging and/or increased nodal connectivity in the brain (e.g. between posterior and anterior aspects of the brain, and/or between occipital-parietal structures and frontal, temporal, and/or limbic structures, for example, as shown by fMRI imaging).

The invention further provides a compound, or a pharmaceutically acceptable salt or prodrug thereof, as defined hereinbefore for use in a method of treating or preventing a neurodegenerative disease in a human subject by reducing glycosphingolipid concentration in brain tissue of the subject, whereby the concentration of glucosylceramide (GL-1) in the cerebrospinal fluid (CSF) of the subject is reduced by at least 30%, wherein the neurodegenerative disease is selected from Parkinson's disease (PD) and dementia with Lewy Bodies (DLB), and wherein the subject does not have (e.g. has not been diagnosed as having, or being at risk of having) a lysosomal storage disease.

In embodiments, said method of treating or preventing is as defined hereinbefore.

The invention also provides the use of a compound, or a pharmaceutically acceptable salt or prodrug thereof, as defined hereinbefore in the manufacture of a medicament for use in a method of treating or preventing a neurodegenerative disease in a human subject by reducing glycosphingolipid concentration in brain tissue of the subject, whereby the concentration of glucosylceramide (GL-1) in the cerebrospinal fluid (CSF) of the subject is reduced by at least 30%, wherein the neurodegenerative disease is selected from Parkinson's disease (PD) and dementia with Lewy Bodies (DLB), and wherein the subject does not have (e.g. has not been diagnosed as having, or being at risk of having) a lysosomal storage disease. In embodiments, said method of treating or preventing is as defined hereinbefore.

The invention further provides a compound, or a pharmaceutically acceptable salt or prodrug thereof, as defined hereinbefore for use in a method of treating or preventing a neurodegenerative disease in a human subject by reducing glycosphingolipid concentration in brain tissue of the subject, whereby the concentration of glucosylceramide (GL-1) in the cerebrospinal fluid (CSF) of the subject is reduced by at least 30%, wherein the neurodegenerative disease is selected from Parkinson's disease (PD) and dementia with Lewy Bodies (DLB), and wherein the subject is a heterozygous carrier of one or more glucocerebrosidase 1 gene (GBA1) mutations (e.g. L444P and/or N370S). In embodiments, said method of treating or preventing is as defined hereinbefore.

In a further aspect, the invention provides the use of a compound, or a pharmaceutically acceptable salt or prodrug thereof, as defined hereinbefore in the manufacture of a medicament for use in a method of treating or preventing a neurodegenerative disease in a human subject by reducing glycosphingolipid concentration in brain tissue of the subject, whereby the concentration of glucosylceramide (GL-1) in the cerebrospinal fluid (CSF) of the subject is reduced by at least 30%, wherein the neurodegenerative disease is selected from Parkinson's disease (PD) and dementia with Lewy Bodies (DLB), and wherein the subject is a heterozygous carrier of one or more glucocerebrosidase 1 gene (GBA1) mutations (e.g. L444P and/or N370S). In embodiments, said method of treating or preventing is as defined hereinbefore.

Additional features and advantages of methods and compounds disclosed herein will be apparent from the following detailed description.

### BRIEF DESCRIPTION OF THE DRAWINGS

FIG. 1 shows the disposition of participants in part 1 of the clinical trial. Two rest of world (“ROW”) participants permanently discontinued the study after receiving Compound 2 and post-Week 4 (primary analysis period) due to adverse effects. One participant (low-dose) discontinued due to confusional state, and one participant (high-dose) due to a panic attack.

FIG. 2 depicts the study design, including secondary and exploratory endpoints. All participants were followed every 4 weeks for a maximum of 36 weeks (up to 52 weeks for Japanese participants). Dose escalation in sequential cohorts was performed when safety and tolerability were demonstrated after data review when all participants completed the first 4-week course of therapy.

FIG. 3 shows the mean plasma concentration of Compound 2 observed in study participants just before treatment administration (C_{trough}) during repeat dosing over 8 weeks. Results are shown for Japanese and ROW participants who received low-, mid-, and high-dose Compound 2. Dashed line indicates the lower limit of quantification (LLOQ=0.5 ng/mL).

FIG. 4 shows the mean percent change from baseline in plasma (A) and CSF (B) GL-1 levels in Japanese and ROW participants who received placebo or were treated with Compound 2 (low-, mid-, or high-dose) in part 1 of the trial. GL-1 levels were assessed in plasma samples collected at baseline, end of Week 2 (dark grey bars; left-hand bar of each pair), and end of Week 4 (light grey bars; right-hand bar of each pair), and in CSF samples collected at baseline and end of Week 4. ^{a}Week 4 CSF sample was not collected for 1 participant from the ROW population.

FIG. 5 shows mean GL-1 levels at baseline (dark grey bars; left-hand bar of each pair) and Week 4 (light grey bars; right-hand bar of each pair) in plasma (A) and CSF (B) in Japanese and ROW participants who received placebo or Compound 2 (low-, mid-, or high-dose) in part 1 of the trial. ^{a}Week 4 CSF sample was not collected for 1 participant from the ROW population.

### DEFINITIONS

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present invention, exemplary methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present disclosure will employ, unless otherwise indicated, conventional techniques of tissue culture, immunology, molecular biology, microbiology, cell biology, and recombinant DNA, which are within the skill of the art. See, e.g., Michael R. Green and Joseph Sambrook, Molecular Cloning (4^{th} ed., Cold Spring Harbor Laboratory Press 2012); the series Ausubel et al. eds. (2007) Current Protocols in Molecular Biology; the series Methods in Enzymology (Academic Press, Inc., N.Y.); MacPherson et al. (1991) PCR 1: A Practical Approach (TRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (TRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3d edition (Cold Spring Harbor Laboratory Press (2002)); Sohail (ed.) (2004) Gene Silencing by RNA Interference: Technology and Application (CRC Press).

All numerical designations, e.g. pH, temperature, time, concentration, molecular weight, etc., including ranges, are approximations which are varied (+) or (−) by increments of 0.1 or 1.0, where appropriate. It is to be understood, although not always explicitly stated that all numerical designations are preceded by the term “about”. The term “about” in connection with any numerical value designates a variability about that value within the conventional range. For example, the numerical value may vary by ±10%, ±5%, ±1.0%, or ±0.5%. It also is to be understood, although not always explicitly stated, that the reagents described herein are merely exemplary and that equivalents of such are known in the art.

For the avoidance of doubt, any disclosure of a numerical range, e.g. “up to X” amount, is intended to include the upper numerical limit X. Therefore, a disclosure of “up to 60 mg” includes 60 mg. Analogously, any disclosure of a numerical range is also intended to include the lower numerical limit, e.g. “from A to”, or “at least A”. Therefore, a disclosure of “from 5 mg to 50 mg” or “at least 5 mg” includes 5 mg.

As used in the specification and claims, the singular form “a”, “an”, and “the” include plural references unless the context clearly dictates otherwise. For example, the term “a cell” includes a plurality of cells, including mixtures thereof. Unless specifically stated or obvious from context, as used herein, the term “or” is understood to be inclusive. The term “including” is used herein to mean, and is used interchangeably with, the phrase “including but not limited to”. The term “and/or” is used to include all combinations of one or more items of a list in which the term is used. For example, “features A, B, and/or C” encompasses each of: “A”, “B”, “C”, “A and B”, “A and C”, “B and C”, and “A, B, and C”.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention. Use of the term “comprising” herein is intended to encompass “consisting essentially of” and “consisting of”.

A “subject,” “individual”, or “patient” is used interchangeably herein, and refers to a human.

“Administering” is defined herein as a means of providing an agent or a composition containing the agent to a subject in a manner that results in the agent being inside the subject's body. Such an administration can be by any route including, without limitation, oral, transdermal (e.g. vagina, rectum, oral mucosa), by injection (e.g. subcutaneous, intravenous, parenterally, intraperitoneally, into the CNS), or by inhalation (e.g. oral or nasal). Pharmaceutical preparations are, of course, given by forms suitable for each administration route.

“Treating” or “treatment” of a disease includes: (1) inhibiting the disease, i.e. arresting or reducing the development of the disease or its clinical symptoms; and/or (2) relieving the disease, i.e. causing regression of the disease or its clinical symptoms.

“Preventing” or “prevention” of a disease includes preventing the disease, i.e. causing the clinical symptoms of the disease not to develop in a patient that may be predisposed to the disease but does not yet experience or display symptoms of the disease.

As used herein, the phrase “in the treatment or prevention of” (such as in the phrase “in the treatment or prevention of pain”) is meant to be equivalent to the phrase “in a method of treating or preventing” (such as in the phrase “in a method of treating or preventing pain”).

Being “at risk of having” a disease (e.g. a patient being at risk of having a lysosomal storage disease) refers to a patient who may be predisposed to the disease, for instance, the patient may have history of disease in their family lineage or the presence of genetic mutations associated with the disease. A patient at risk of having a disease has not yet developed all or some of the characteristic pathologies of the disease.

The term “suffering” as it relates to the term “treatment” refers to a patient or individual who has been diagnosed with or is predisposed to the disease. A patient may also be referred to being “at risk of suffering” from a disease because of a history of disease in their family lineage or because of the presence of genetic mutations associated with the disease.

The term “heterozygous” refers to a subject who possesses two alleles which are non-identical. A “heterozygous mutation” means either a gene in which a single allele carries one or more mutations and the other allele carries no mutations, or a gene in which both alleles carry one or more mutations, wherein the mutations differ between the two alleles (“compound heterozygous”).

An “effective amount” or “therapeutically effective amount” is an amount sufficient to effect the required therapeutic or physiological outcome, namely for reducing glycosphingolipid concentration in brain tissue of a subject, for instance as part of treating or preventing a neurodegenerative disease selected from Parkinson's disease (PD) and dementia with Lewy Bodies (DLB). An effective amount can be administered in one or more administrations, applications, or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including, for example, the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve reduction in GL-1 level in the CSF of the subject, thereby verifying reduction of glycosphingolipid concentration in the brain tissue of the subject.

As used herein, the term “pharmaceutically acceptable excipient” encompasses any of the standard pharmaceutical excipients, including carriers such as a phosphate buffered saline solution, water, and emulsions, such as an oil/water or water/oil emulsion, and various types of wetting agents. Pharmaceutical compositions also can include stabilizers and preservatives. For examples of carriers, stabilizers, and adjuvants, see Remington's Pharmaceutical Sciences (20th ed., Mack Publishing Co. 2000).

As used herein, the term “prodrug” means a pharmacological derivative of a parent drug molecule that requires biotransformation, either spontaneous or enzymatic, within the organism to release the active drug. For example, prodrugs are variations or derivatives of the quinuclidine compounds described herein that have groups cleavable under certain metabolic conditions, which, when cleaved, become the quinuclidine compounds described herein, e.g. a compound of Formula I. Such prodrugs then are pharmaceutically active in vivo when they undergo solvolysis under physiological conditions or undergo enzymatic degradation. Prodrug compounds herein may be called single, double, triple, etc., depending on the number of biotransformation steps required to release the active drug within the organism, and the number of functionalities present in a precursor-type form. Prodrug forms often offer advantages of solubility, tissue compatibility, or delayed release in the mammalian organism (Bundgard, Design of Prodrugs, pp. 7-9, 21-24, Elsevier, Amsterdam 1985; and Silverman, “The Organic Chemistry of Drug Design and Drug Action” pp. 352-401, Academic Press, San Diego, Calif., 1992).

Prodrugs commonly known in the art include well-known acid derivatives, such as, for example, esters prepared by reaction of acid compounds with a suitable alcohol, amides prepared by reaction of acid compounds with an amine, basic groups reacted to form an acylated base derivative, etc. Other prodrug derivatives may be combined with other features disclosed herein to enhance bioavailability. As such, those of skill in the art will appreciate that certain of the presently disclosed compounds having, for example, free amino or hydroxy groups can be converted into prodrugs. Prodrugs include compounds having an amino acid residue, or a polypeptide chain of two or more (e.g. two, three, or four) amino acid residues which are covalently joined through peptide bonds to free amino, hydroxy, or carboxylic acid groups of the presently disclosed compounds. The amino acid residues include the 20 naturally occurring amino acids commonly designated by three letter symbols and also include 4-hydroxyproline, hydroxylysine, demosine, isodemosine, 3-methylhistidine, norvalin, beta-alanine, gamma-aminobutyric acid, citrulline, homocysteine, homoserine, ornithine, and methionine sulfone. Prodrugs also include compounds having a carbonate, carbamate, amide, or alkyl ester moiety covalently bonded to any of the above substituents disclosed herein.

As used herein, the term “pharmaceutically acceptable salt” means a pharmaceutically acceptable acid addition salt or a pharmaceutically acceptable base addition salt of a currently disclosed compound that may be administered without any resultant substantial undesirable biological effect(s) or any resultant deleterious interaction(s) with any other component of a pharmaceutical composition in which it may be contained.

As used herein, the term “free base” means the compound referenced per se, e.g. not in the form of a salt or prodrug. Reference to, e.g., a “dose of . . . 5 mg (measured as the equivalent amount of free base)” means that there is an amount of compound present, whether in the form of the free base or a salt or prodrug, which corresponds to the same molar amount of 5 mg of the freebase, i.e. the compound in salt form would have a mass of greater than 5 mg. Unless otherwise indicated, an amount of an active compound for administration (whether administered as a free base or as a salt form) refers to or is based on the amount of the compound in free base form.

As used herein, the term “C₁₋₆-alkyl” means a saturated linear or branched free radical consisting essentially of 1 to 6 carbon atoms and a corresponding number of hydrogen atoms. Exemplary C₁₋₆-alkyl groups include methyl, ethyl, n-propyl, isopropyl, n-butyl, isobutyl, etc. Other C₁₋₆-alkyl groups will be readily apparent to those of skill in the art given the benefit of the present disclosure. The terms “C₁₋₃-alkyl”, “C₁₋₄-alkyl”, etc., have equivalent meanings, i.e. saturated linear or branched free radical consisting essentially of 1 to 3 (or 4) carbon atoms and a corresponding number of hydrogen atoms.

As used herein, the term “C₂₋₆-alkenyl” means an unsaturated linear or branched free radical consisting essentially of 2 to 6 carbon atoms and a corresponding number of hydrogen atoms, which free radical comprises at least one carbon-carbon double bond. Exemplary C₂₋₆-alkenyl groups include ethenyl, prop-1-enyl, prop-2-enyl, isopropenyl, but-1-enyl, 2-methyl-prop-1-enyl, 2-methyl-prop-2-enyl, etc. Other C₂₋₆-alkenyl groups will be readily apparent to those of skill in the art given the benefit of the present disclosure.

As used herein, the term “C₂₋₆-alkynyl” means an unsaturated linear or branched free radical consisting essentially of 2 to 6 carbon atoms and a corresponding number of hydrogen atoms, which free radical comprises at least one carbon-carbon triple bond. Exemplary C₂₋₆-alkynyl groups include ethynyl, prop-1-ynyl, prop-2-ynyl, but-1-ynyl, 3-methyl-but-1-ynyl, etc. Other C₂₋₆-alkynyl groups will be readily apparent to those of skill in the art given the benefit of the present disclosure.

As used herein, the term “C₁₋₆-alkyloxy” means a saturated linear or branched free radical consisting essentially of 1 to 6 carbon atoms (and a corresponding number of hydrogen atoms) and an oxygen atom. A C₁₋₆-alkyloxy group is attached via the oxygen atom. Exemplary C₁₋₆-alkyloxy groups include methyloxy, ethyloxy, n-propyloxy, isopropyloxy, n-butyloxy, isobutyloxy, etc. Other C₁₋₆-alkyloxy groups will be readily apparent to those of skill in the art given the benefit of the present disclosure. The terms “C₁₋₃-alkyloxy”, “C₁₋₄-alkyloxy”, and the like, have an equivalent meaning, i.e. a saturated linear or branched free radical consisting essentially of 1 to 3 (or 4) carbon atoms (and a corresponding number of hydrogen atoms) and an oxygen atom, wherein the group is attached via the oxygen atom.

As used herein, the term “C₂₋₆-alkenyloxy” means an unsaturated linear or branched free radical consisting essentially of 2 to 6 carbon atoms (and a corresponding number of hydrogen atoms) and an oxygen atom, which free radical comprises at least one carbon-carbon double bond. A C₂₋₆-alkenyloxy group is attached via the oxygen atom. An exemplary C₂₋₆-alkenyloxy group is ethenyloxy; others will be readily apparent to those of skill in the art given the benefit of the present disclosure.

As used herein, the term “C₂₋₆-alkynyloxy” means an unsaturated linear or branched free radical consisting essentially of 2 to 6 carbon atoms (and a corresponding number of hydrogen atoms) and an oxygen atom, which free radical comprises at least one carbon-carbon triple bond. A C₂₋₆-alkenyloxy group is attached via the oxygen atom. An exemplary C₂₋₆-alkenyloxy group is ethynyloxy; others will be readily apparent to those of skill in the art given the benefit of the present disclosure.

As used herein, the term “heteroaryl” means an aromatic free radical having 5 or 6 atoms (i.e. ring atoms) that form a ring, wherein 1 to 5 of the ring atoms are carbon and the remaining 1 to 5 ring atom(s) (i.e. hetero ring atom(s)) is selected independently from the group consisting of nitrogen, sulfur, and oxygen. Exemplary 5-membered heteroaryl groups include furyl, thienyl, thiazolyl (e.g. thiazol-2-yl), pyrazolyl, isothiazolyl, oxazolyl, isoxazolyl, pyrrolyl, triazolyl, imidazolyl, oxadiazolyl, and thiadiazolyl. Exemplary 6-membered heteroaryl groups include pyridyl, pyrimidyl, pyrazinyl, pyridazinyl, 1,2,4-triazinyl, benzoxazolyl, benzothiazolyl, benzisothiazolyl, benzisoxazolyl, benzimidazolyl, etc. Other heteroaryl groups will be readily apparent to those of skill in the art given the benefit of the present disclosure. In general, the heteroaryl group typically is attached to the main structure via a carbon atom. However, those of skill in the art will realize that certain other atoms, e.g. hetero ring atoms, can be attached to the main structure.

As used herein, the term “aryl” means an aromatic free radical having 5 or 6 atoms (i.e. ring atoms) that form a ring, wherein all of the ring atoms are carbon. An exemplary aryl group is phenyl.

As used herein, the term “aliphatic” means a non-aromatic compound containing carbon and hydrogen atoms, e.g. containing 1 to 9 carbon atoms. Aliphatic compounds may be straight-chained or branched, may contain one or more ring structures, and may contain one or more carbon-carbon double bonds (provided that the compound does not contain an unsaturated ring structure having aromatic character). Examples of aliphatic compounds include ethane, propylene, cyclobutane, cyclohexadiene, etc.

As used herein, the terms “halo” and “halogen” mean fluorine, chlorine, bromine, or iodine. These terms are used interchangeably and may refer to a halogen free radical group or to a halogen atom as such. Those of skill in the art will readily be able to ascertain the identification of which in view of the context in which this term is used in the present disclosure.

As used herein, the term “cyano” means a free radical having a carbon atom linked to a nitrogen atom via a triple bond. The cyano radical is attached via its carbon atom.

As used herein, the term “nitro” means an NO₂ radical which is attached via its nitrogen atom.

As used herein, the terms “hydroxy” and “hydroxyl” mean an OH radical which is attached via its oxygen atom. The term “thio” means an SH radical which is attached via its sulphur atom.

As used herein, the term “amino” means a free radical having a nitrogen atom and 1 or 2 hydrogen atoms. As such, the term “amino” generally refers to primary and secondary amines. In that regard, as used herein, a tertiary amine is represented by the general formula RR′N—, wherein R and R′ are carbon radicals that may or may not be identical. Nevertheless, the term “amino” generally may be used herein to describe a primary, secondary, or tertiary amine, and those of skill in the art will readily be able to ascertain the identification of which in view of the context in which this term is used in the present disclosure.

As used herein, the term “oxo” means an oxygen radical which is attached via a double bond. Where an atom bonded to this oxygen is a carbon atom, the bond is a carbon-oxygen double bond which may be denoted as —(C═O)— and which may be referred to as a carbonyl (e.g. a ketone).

The term “optionally substituted” used herein refers to the possibility of a particular group being either substituted (e.g. where a hydrogen radical bonded to a carbon atom may be replaced by a non-hydrogen radical of appropriate valence) or not substituted (i.e. where a hydrogen radical is not replaced by a non-hydrogen radical).

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

The following abbreviations are used herein:
AE Adverse event
AESI Adverse event of special interest
AMTS Abbreviated mental test score
BDI-II Beck's Depression Inventory-II
br Broad signal
CDI Carbonyldiimidazole
cDNA Complimentary deoxyribonucleic acid
CNS Central Nervous System
CSF Cerebrospinal fluid
CV % Geometric coefficient of variation
d Doublet
DAT Dopamine transporter
dd Doublet of doublets
DLB Dementia with Lewy Bodies
DMF Dimethylformamide
DNA Deoxyribonucleic acid
DOPA Dihydroxyphenylalanine
equiv. Equivalents
fMRI Functional Magnetic Resonance Imaging
GBA1 Glucocerebrosidase 1 gene
GBA-PD GBA-associated Parkinson's disease
GCase Glucocerebrosidase
GCS Glucosylceramide synthase
GD Gaucher disease
GL-1 Glucosylceramide
GlcSph Glucosylsphingosine (also lyso-GL1)
HPLC High pressure/performance liquid chromatography
IPA Isopropyl alcohol
IQCODE Informant questionnaire on cognitive decline in the elderly
IQR Interquartile range
J Coupling constant
LCMS Liquid chromatography mass spectrometry
LC/MS/MS Liquid chromatography tandem mass spectrometry
LC/ESI/DMS Liquid chromatography electrospray ionisation differential ion mobility spectrometry
LLOQ Lower limit of quantification
LP Lumbar puncture
LRRK2 Leucine rich repeat kinase 2 gene
lyso-GL1 Glucosylsphingosine (also GlcSph)
m Multiplet
MDS Movement Disorder Society
MDS-UPDRS Movement Disorder Society-Unified Parkinson's Disease Rating Scale
M-EDL Motor Aspects of Experiences of Daily Living (MDS-UPDRS Part II)
MedDRA Medical Dictionary for Regulatory Activities
MMSE Mini mental state examination
MoCA Montreal Cognitive Assessment
mRNA Messenger ribonucleic acid
PD Parkinson's disease
PET Positron emission tomography
PO By mouth (oral administration)
ppm Parts per million
qd Once per day
RB Round-bottomed
RBD Rapid eye movement sleep behaviour disorder
ROW Rest of the world (here, any country except Japan)
s Singlet
SD Standard deviation
SPECT Single-photon emission computed tomography
TBME Tert-Butyl Methyl Ether
THF Tetrahydrofuran
TMT Trail-making test
ULN Upper limit of normal
UPDRS Unified Parkinson's Disease Rating Scale
UPLCMS Ultra performance liquid chromatography mass spectrometry

The reference works, patents, patent applications, and scientific literature, and other printed publications that are mentioned or referred to herein are hereby incorporated by reference in their entirety.

### DETAILED DESCRIPTION

The quinuclidine compounds, and pharmaceutical compositions containing them, described herein are useful in therapy, where reducing glycosphingolipid concentration in brain tissue of a human subject can restore a normal glycosphingolipid environment or can prevent or slow the development of an abnormal (e.g. toxic) glycosphingolipid environment that otherwise contributes towards the pathogenesis of a disease state. This may be achieved by inhibiting the biosynthesis of the glycosphingolipids and/or their substrates through the methods disclosed herein. Without being bound by any particular theory, it is postulated that inhibition of substrate accumulation equilibrates biosynthesis with the impaired catabolism associated with glucocerebrosidase, for instance as a result of a GBA1 mutation.

### Therapeutic Methods

Thus, in a first aspect the invention provides a method of treating or preventing a neurodegenerative disease in a human subject by reducing glycosphingolipid concentration in brain tissue of the subject, whereby the concentration of glucosylceramide (GL-1) in the cerebrospinal fluid (CSF) of the subject is reduced by at least about 30%, wherein the neurodegenerative disease is selected from Parkinson's disease (PD) and dementia with Lewy Bodies (DLB), and wherein the subject does not have (e.g. has not been diagnosed as having, or being at risk of having) a lysosomal storage disease; the method comprising administering to the subject an effective amount of a quinuclidine compound as defined herein (e.g. Compound 2). In embodiments, the subject does not have (e.g. has not been diagnosed as having, or being at risk of having) a lysosomal storage disease selected from Gaucher disease, Fabry disease, and Krabbe disease. In particular, the subject does not have (e.g. has not been diagnosed as having, or being at risk of having) Gaucher disease. A subject can be determined to have Gaucher disease as defined by clinical signs and symptoms, which may include one or more of hepatosplenomegaly, cytopenia, and skeletal disease. Additionally or alternatively, a subject can be determined to have Gaucher disease as defined by marked deficiency of GCase activity.

In some embodiments, the subject is not a homozygous carrier of a GBA1 mutation and/or is not a compound heterozygous carrier. In other embodiments, the subject is a heterozygous carrier of one or more GBA1 mutations. The one or more gene (GBA1) mutations may, for instance, be selected from severe mutations (e.g. those denoted as a “severe GBA mutation” in Table 1, such as L444P) and other (i.e. non-severe) GBA1 mutations (e.g. those denoted as an “other GBA mutation” in Table 1, such as N370S). In embodiments, the one or more GBA1 mutations are selected from L444P, 84GG, A456P, R120W, D409H, E235A, E340A, N370S, E326K, R496C, G193W, T369M, R496H, and S271G. For example, the subject is a carrier of a L444P mutation, and optionally one or more further GBA1 mutations. In another example, the subject is a carrier of a N370S mutation, and optionally one or more further GBA1 mutations.

In a second aspect, the invention provides a method of treating or preventing a neurodegenerative disease in a human subject by reducing glycosphingolipid concentration in brain tissue of the subject, whereby the concentration of glucosylceramide (GL-1) in the cerebrospinal fluid (CSF) of the subject is reduced by at least about 30%, wherein the neurodegenerative disease is selected from Parkinson's disease (PD) and dementia with Lewy Bodies (DLB), and wherein the subject is a heterozygous carrier of one or more glucocerebrosidase 1 gene (GBA1) mutations; the method comprising administering to the subject an effective amount of a quinuclidine compound as defined herein (e.g. Compound 2). In embodiments, the subject does not have (e.g. has not been diagnosed as having, or being at risk of having) a lysosomal storage disease, e.g. Gaucher disease.

The one or more gene (GBA1) mutations may, for instance, be selected from severe mutations (e.g. those denoted as a “severe GBA mutation” in Table 1, such as L444P) and other (i.e. non-severe) GBA1 mutations (e.g. those denoted as an “other GBA mutation” in Table 1, such as N370S). In embodiments, the one or more GBA1 mutations are selected from L444P, 84GG, A456P, R120W, D409H, E235A, E340A, N370S, E326K, R496C, G193W, T369M, R496H, and S271G. For example, the subject is a carrier of a L444P mutation, and optionally one or more further GBA1 mutations. In another example, the subject is a carrier of a N370S mutation, and optionally one or more further GBA1 mutations.

In a third aspect, the present invention provides a therapeutic method of reducing glycosphingolipid concentration in brain tissue of a human subject in need thereof, whereby the concentration of glucosylceramide (GL-1) in the cerebrospinal fluid (CSF) of the subject is reduced as a result of the method, wherein the subject does not have (e.g. has not been diagnosed as having, or being at risk of having) a lysosomal storage disease; the method comprising administering to the subject an effective amount of a quinuclidine compound as defined herein (e.g. Compound 2). In embodiments, the method results in a reduction in GL-1 concentration in CSF of at least about 30%.

Further aspects of the invention provide a quinuclidine compound as defined herein (e.g. Compound 2) for use in a method of the invention. Yet further aspects provide the use of a quinuclidine compound as defined herein (e.g. Compound 2) in the preparation of a medicament for use in a method of the invention.

### Subjects to be Treated

The present invention provides therapeutic methods for reducing glycosphingolipid concentration in brain tissue of a human subject in need thereof, whereby the concentration of GL-1 in the CSF of the subject is reduced as a result of the method. It is envisaged that the effect of reducing glycosphingolipid concentration in brain tissue can either be used to maintain or restore a normal glycosphingolipid environment, or to avoid, suspend, or reverse disease pathogenesis associated with abnormal (e.g. heightened) glycosphingolipid concentration in brain tissue.

In particular aspects and embodiments, the subject does not have (e.g. has not been diagnosed as having, or being at risk of having) a lysosomal storage disease such as, for example, Gaucher, Fabry, Krabbe, G_{M1}-gangliosidosis, G_{M2} Activator deficiency, Tay-Sachs, or Sandhoff. In particular embodiments, the subject does not have (e.g. has not been diagnosed as having, or being at risk of having) Gaucher disease. In embodiments, the subject does not carry a leucine rich repeat kinase 2 gene (LRRK2) G2019S mutation.

### GBA Mutations

In particular aspects and embodiments, the subject is a heterozygous carrier of one or more glucocerebrosidase 1 gene (GBA1) mutations, and therefore does not suffer from a lysosomal storage disease such as, for example, Gaucher disease. Lysosomal storage diseases can be characterized by biallelic mutations (homozygous or compound heterozygous), e.g. biallelic GBA1 mutations.

GBA1 mutations are the most common genetic risk factor for developing Parkinson's disease (PD). Known severe GBA1 mutations include L444P, 84G>GG, and A456P, whilst other known non-severe GBA1 mutations include D409H, P409V, E235A, E340A, N370S, E326K, R496C, G193W, T369M, R496H, and S271G.

In embodiments, the subject to be treated by a method of the invention has one or more mutations in GBA1 selected from the list in Table 1. In one embodiment, the mutation is a mutation denoted as a “severe GBA mutation” in Table 1. In another embodiment, the mutation is a mutation denoted as an “other GBA mutation” in Table 1. A particular example of a severe GBA mutation is L444P. A particular example of an “other GBA mutation”, i.e. a non-severe GBA mutation, is N370S.

In embodiments, the subject to be treated has one or more mutations in GBA1 selected from L444P, 84G>GG, A456P, D409H, E235A, E340A, N370S, E326K, R496C, G193W, T369M, R496H, and S271G. In other embodiments, the subject to be treated has one or more mutations in GBA1 selected from 84G>GG, A456P, D409H, E235A, E340A, N370S, E326K, R496C, G193W, T369M, R496H, and S271G. In other embodiments, the subject to be treated has one or more mutations in GBA1 selected from 84G>GG, A456P, E235A, E340A, N370S, E326K, R496C, G193W, T369M, R496H, and S271G. In other embodiments, the subject to be treated has one or more mutations in GBA1 selected from L444P, N370S, and E326K. In other embodiments, the subject to be treated has a L444P mutation. In other embodiments, the subject to be treated has a N370S mutation. In other embodiments, the subject to be treated has a E326K mutation.

In embodiments, the subject to be treated has a mutation in GBA1 selected from E326K and T369M and the subject also has a history of RBD, e.g. as determined by a history of documented polysomnography or by RBD screening questionnaire. In other embodiments, the subject to be treated has a mutation in GBA1 selected from E326K and T369M and the subject also has at least one further mutation in GBA1, optionally wherein the further mutation is selected from L444P, 84G>GG, A456P, D409H, E235A, E340A, N370S, R496C, G193W, R496H, and S271G. In particular embodiments, the further mutation is selected from L444P and N370S.

In one embodiment, the subject to be treated does not have a mutation at L444, e.g. the subject does not have a L444P mutation in GBA1. In one embodiment, the subject does not have a mutation at D409, e.g. the subject does not have a D409V mutation in GBA1.

In certain other embodiments, the subject does not have a deleterious GBA1 mutation, e.g. the gene functions substantially normally in that it encodes a protein with essentially the same structure, activity, and/or tissue levels and distribution as the protein encoded by the wild-type gene. Wild-type GBA1 sequences are known in the art and include GenBank accession number NM_000157.3 (mRNA).

### Subjects Having Pre-Existing Conditions, Medications, or Treatment History

The methods disclosed herein may be deemed unsuitable for certain patient groups, for example those having certain pre-existing conditions, those with current or past treatment with some medications, and those with history of treatments, such as surgical treatments, as assessed and described herein. A prescribing physician will be qualified to decide whether a subject's particular condition(s), and/or current or past medication(s) affect their suitability for undergoing methods of treatment according to the methods disclosed herein.

In embodiments, the subject to be treated does not have past surgical history of deep brain stimulation.

In embodiments, the subject to be treated does not have severe depression, e.g. as measured by BDI-II above 28. Additionally or alternatively, the subject to be treated does not have a history of a major affective disorder (e.g. has not been diagnosed as having a major affective disorder) prior to treatment. In embodiments, the subject to be treated does not have a history of drug or alcohol abuse prior to treatment. Preferably, the subject to be treated does not have any such history of major affective disorder or drug or alcohol abuse within 1 year prior to treatment.

In embodiments, the subject to be treated has not taken or been administered any medication specifically used for treating memory dysfunction (e.g. cholinesterase inhibitors, or memantine) within 30 days or 5 half-lives prior to treatment, whichever is longer. In embodiments, the subject to be treated has not taken or been administered any medication selected from antipsychotics, modafinil, armodafinil, metoclopramide, alpha-methyldopa, methylphenidate, reserpine, or amphetamine derivatives (including medications which have amphetamine metabolites, e.g. MAOB inhibitors such as selegiline), within 6 months prior to treatment. In embodiments, the subject to be treated has not taken or been administered one or more strong or moderate inducers or inhibitors of CYP3A4 within 30 days or 5 half-lives prior to treatment, whichever is longer. In embodiments, the subject to be treated is not undergoing treatment with anticoagulants (e.g. warfarin (coumadin), or heparin) at the time of treatment. In embodiments, the subject to be treated has not taken or been administered an investigational medicinal product, e.g. ambroxol, within 3 months or 5 half-lives prior to treatment, whichever is longer.

In embodiments, the subject to be treated does not have a marked baseline prolongation of QT/QTc interval as measured by ECG, e.g. a QTc interval greater than 450 msec in male subjects and a QTc interval greater than 470 msec in female subjects. In embodiments, the subject to be treated is not taking or being administered medications that prolong the QT/QTc interval. The QT/QTc interval is the time from ECG Q wave to the end of the T wave or corrected T wave corresponding to electrical systole.

In embodiments, the subject to be treated does not have liver enzymes (ALT/AST or total bilirubin greater than 2 times the ULN at the time of treatment. In embodiments, the subject to be treated does not have Gilbert's disease. In embodiments, the subject to be treated does not have renal insufficiency, e.g. as defined by creatinine greater than 1.5 times ULN at the time of treatment. In embodiments, the subject to be treated does not have (e.g. has not been diagnosed as having) any of hepatitis B, hepatitis C, and human immunodeficiency virus 1 or 2.

In embodiments, the subject to be treated does not have a medical disorder and/or clinically relevant finding (e.g. by physical examination, medical history, or laboratory assessment) selected from heart failure, hypokalemia, prohibitive lumbar spinal disease, bleeding diathesis, or clinically significant coagulopathy or thrombocytopenia.

In embodiments, the subject to be treated does not have a cortical cataract greater than one quarter of the lens circumference (Grade cortical cataract-2) or a posterior subcapsular cataract greater than 2 mm (Grade posterior subcapsular cataract-2), according to World Health Organization Grading. In embodiments, the subject to be treated has not taken or been administered, or is not taking or being administered at the time of treatment, potentially cataractogenic medications. Cataractogenic medications include a chronic regimen (e.g. more frequently than every 2 weeks) of any dose or route of corticosteroids, and any medication that may cause cataract or worsen the vision of a subject with cataract (e.g. glaucoma medications) according to the prescribing information.

Parkinson's Disease and Dementia with Lewy Bodies

The methods disclosed herein are, in particular, useful in the therapeutic treatment of a neurodegenerative disease selected from Parkinson's disease (PD) and dementia with Lewy Bodies (DLB), where an abnormal glycosphingolipid environment may lead to impaired lysosomal function, misprocessing of the neuronal protein α-synuclein, and neuronal dysfunction, and thereby contribute to pathogenesis.

In particular aspects and embodiments, the subject is suffering from (e.g. has been diagnosed as having), or has been determined to be at risk of suffering from, Parkinson's disease (PD) or dementia with Lewy Bodies (DLB). In one embodiment, the subject is suffering from (e.g. has been diagnosed as having), or has been determined to be at risk of suffering from, PD. In particular embodiments, the subject does not have (e.g. has not been diagnosed as having, or has not been determined as being at risk of suffering from) secondary or atypical PD. Thus, in one embodiment, the subject does not have (e.g. has not been diagnosed as having, or has not been determined as being at risk of suffering from) PD resulting from one or more drugs. In an alternative embodiment, the subject does not have (e.g. has not been diagnosed as having, or has not been determined as being at risk of suffering from) PD resulting from one or more toxins. In a further embodiment, the subject does not have (e.g. has not been diagnosed as having, or has not been determined as being at risk of suffering from) PD resulting from one or more drugs and one or more toxins. In particular embodiments, the subject to be treated does not have a diagnosed structural abnormality, e.g. as determined by MRI without contrast, that is a possible aetiology of PD related signs and symptoms. In another embodiment, the subject is suffering from (e.g. has been diagnosed as having), or has been determined to be at risk of suffering from, DLB.

Subjects with Parkinson's disease and dementia with Lewy Bodies can present with an impairment of neural function, e.g. cognitive function, autonomic function, and/or motor function, depending on the stage of disease progression. Administration of quinuclidine compounds as described herein can result in the improvement of neural function in subjects, e.g. in subjects exhibiting cognitive impairment (as part of, for instance, a Parkinson's disease or dementia with Lewy Bodies disease pathology). Accordingly, in certain embodiments of the present methods, a quinuclidine compound as described herein is administered to a subject having impaired neural (e.g. neurologic) function. In particular embodiments, administration of the quinuclidine compound is initiated after the subject has been diagnosed with impaired neural (e.g. neurologic) function. Diagnosis of a cognitive impairment is within the routine skill of a medical practitioner. Cognitive tests are known in the art and can include tests such as the abbreviated mental test score (AMTS), the mini mental state examination (MMSE), informant questionnaire on cognitive decline in the elderly (IQCODE), and the General Practitioner Assessment of Cognition that test for cognitive impairment. These tests can assess impairments in, for example, memory, reasoning skills, problem solving skills, decision making skills, attention span, and language skills.

One such test is the trail making test (TMT), which may be used to evaluate cognitive function in patients. The TMT is one of the most widely used neuropsychological tests, and is a diagnostic tool for assessing general intelligence and cognitive dysfunctions. In part A of the TMT, subjects are asked to connect a cluster of numbers in ascending order. This task is a combination of visual search, and general visual and motor processing speed. Part B presents a sequence which alternates between numbers and letters. Subjects must actively switch between both categories when connecting them in ascending, but alternating, order. Hence, this task is considered to include an executive function component since the subject must actively switch between categories while connecting the symbols.

Another well-known screening tool which may be used to assess mild cognitive impairment is the Montreal Cognitive Assessment (MoCA) (Nasreddine et al., J Am Geriatr Soc 2005; 53: 695-9).

Where a subject has a Parkinson's diagnosis specifically, a well-known scale for assessing and reporting disease progression is the Hoehn and Yahr Stage scale (Hoehn M M, Yahr M D. Parkinsonism: onset, progression and mortality. Neurology 1967; 17: 427-42), which identifies 5 stages of disease progression and can be used to distinguish between early-, mid-, and late-stage Parkinson's disease patients. For instance, early-stage/mild Parkinson's disease is generally considered to correspond to a Hoehn and Yahr Stage of ≤2 (Unilateral involvement only, usually with minimal or no functional disability (Stage 1), or Bilateral or midline involvement without impairment of balance (Stage 2)).

Another notable research tool used in research and in a clinical setting which specifically relates to Parkinson's disease progression is the MDS-UPDRS, which is widely used to support endpoints in PD clinical trials and aims to assess the functional impact of PD on patients' daily lives. The MDS-UPDRS is a revision of the Unified Parkinson's Disease Rating Scale (UPDRS), originally developed in the 1980s, completed by the MDS (Movement Disorder Society) review task force. The MDS-UPDRS was developed to evaluate various aspects of Parkinson's disease, including non-motor and motor experiences of daily living and motor complications (Goetz et al., Mov Disord 2007; 22: 41-7, and Goetz et al., Mov Disord 2008; 23:2129-70). It includes a motor evaluation and characterizes the extent and burden of disease across various populations.

Correlation between MDS-UPDRS and Hoehn and Yahr stage scale has been investigated (Skorvanek M et al., Mov Disord Clin Pract 2017; 4: 536-544). The MDS-UPDRS was designed to discriminate especially mild rather than severe symptoms of the disease, whereas the original UPDRS better discriminates the severe and very severe symptoms. Part II of the MDS-UPDRS measures Motor Aspects of Experiences of Daily Living (M-EDL). Part III of the MDS-UPDRS is a Motor Examination. The range of MDS-UPDRS Parts II+III in patients with early stage/mild PD can typically be from, for example, 10 to 80, or from 20 to 70, or from 20 to 60, or from 25 to 65, or from 30 to 70, or from 35 to 65, or from 35 to 60, or from 20 to 45, or from 35 to 55, or from 35 to 50.

Imaging methods are also available to diagnose cognitive decline. For example, the functional neuroimaging modalities of single-photon emission computed tomography (SPECT) and positron emission tomography (PET) are useful in assessing cognitive dysfunction. Neurological function may also be evaluated using functional magnetic resonance imaging (fMRI). In some aspects, the improvement of neural function is measured by evaluating the cognitive function of the patient. Cognitive deterioration, e.g. associated with mild cognitive impairment, may also be assessed by monitoring different cognitive domains. Cognitive domains include, for example, attention and concentration, executive functions, memory, language, visuo-constructional skills, conceptual thinking, calculations, and orientation. Diagnosis of other impairments associated with Parkinson's disease is also within the routine skill of a medical practitioner. For example, clinical criteria for a diagnosis of Parkinson's disease involve assessing impairments in motor and/or autonomic functions, e.g. slowness of movement (bradykinesia), plus either rigidity, resting tremor, or postural instability. Responsiveness to dopamine (symptomatic treatment) and reduced dopaminergic activity in the basal ganglia can also aid in diagnosing Parkinson's disease.

Accordingly, the invention provides methods for treating or preventing Parkinson's disease in a subject, as described herein. The methods of the invention may be beneficial for subjects who have been diagnosed as being at risk of developing Parkinson's disease due to, for example, a mutation in the subject or the subject's family lineage known to cause Parkinson's disease, but are not yet experiencing the typical symptoms associated with the disease state, e.g. signs of cognitive impairment. In one embodiment, the subject has been diagnosed as being at risk of developing Parkinson's disease, and the methods prevent or delay the onset and/or development of Parkinson's disease in the subject.

In one embodiment, the subject has a diagnosis of Parkinson's disease, and the methods prevent, reduce, or reverse motor dysfunction (e.g. tremor), bradykinesia, rigidity, postural instability, and/or impaired balance.

In one embodiment, the subject has a diagnosis of Parkinson's disease and the subject has at least one of the following characteristics: i) a family history of Parkinson's disease; ii) a baseline Montreal Cognitive Assessment (MoCA) score of ≤26 (for example, from 20 to 25); and iii) a Movement Disorder Society-Unified Parkinson's Disease Rating Scale (MDS-UPDRS) (Part II+III) score of at least 35 (e.g. at least 40, or at least 45). In embodiments, the subject to be treated does not have a MoCA score of less than 20.

In another embodiment, the subject has a diagnosis of early-stage Parkinson's disease characterized by: i) at least two of the following conditions: resting tremor, postural instability, akinesia/hypokinesia, and muscle rigidity; ii) a Hoehn and Yahr Scale stage of ≤2 at baseline; and/or iii) a Parkinson's diagnosis of ≥2 years.

The treatment methods described herein may be applied concurrently with, or in place of, conventional symptomatic therapies for treatment of Parkinson's disease symptoms. In one embodiment, the subject has been treated historically with medications selected from dihydroxyphenylalanine (DOPA) or derivatives thereof (e.g. levodopa/carbidopa), monoamine oxidase B inhibitors (e.g. rasagiline or selegiline), dopamine agonists (e.g. ropinirole, bromocriptine, cabergoline, pergolide, pramipexole, or apomorphine), Catechol-O-methyltransferase inhibitors (e.g. entacapone, tolcapone), anticholinergics (e.g. artane, cogentin), adamantane derivatives, and/or acetylcholinesterase inhibitors (e.g. tacrine, rivastigmine, galantamine, donepezil, or memantine), optionally wherein, when applied concurrently, the subject has been treated with a stable regimen of symptomatic PD medication for ≥30 days (such as for ≥60 days, for example when the symptomatic PD medication is selected from monoamine oxidase B inhibitors such as rasagiline or selegiline) prior to commencing the treatment methods described herein.

The methods described herein can prevent, reduce, or reverse the progression of dementia, including dementia associated with Parkinson's disease (Parkinson's disease dementia) or dementia with Lewy Bodies. Accordingly, the present invention provides a method of treating or preventing dementia with Lewy Bodies, as described herein.

Symptoms of dementia which may be prevented, reduced, or reversed include early symptoms of dementia, such as difficulty remembering recent conversations, names or events, and apathy and depression, as well as later symptoms, such as impaired communication, poor judgment, disorientation, confusion, behavior changes, and difficulty in speaking, swallowing, and/or walking.

The methods described herein can prevent, reduce, or reverse loss of cognitive function, autonomic function, and/or motor function associated with Parkinson's disease or Dementia with Lewy Bodies in a subject. In one embodiment, the loss of neural function comprises loss of cognitive function. In certain embodiments, the method prevents, reduces, or reverses deterioration in cognitive domains in a subject, e.g. the method prevents, reduces, or reverses deterioration in attention and concentration, executive functions, memory (e.g. working memory), language, visuo-constructional skills, conceptual thinking, calculations, orientation, decision making, problem solving, and the like. In one embodiment, the loss of neural function comprises loss of autonomic function.

In an embodiment, the methods described herein are effective to improve cognitive ability or reduce cognitive deficits in the subject as measured by a reduction in the time taken to complete the trail-making test (TMT), TMT-A and/or TMT-B, a reduction in the difference between TMT-A time and TMT-B time (TMT-B−TMT-A), for example, a reduction of at least 10%, or at least 20%, or at least 30%, or at least 40%, or at least 50% (e.g. wherein TMT-A decreases by 5-20%, and/or TMT-B decreases by 25-30%, and/or [TMT-B−TMT-A] decreases by 25-30%).

### Reducing Glycosphingolipid Levels in the CNS

In restoring a normal glycosphingolipid environment, or reducing or avoiding the development of an abnormal (e.g. toxic) glycosphingolipid environment associated with disease pathogenesis, the present methods reduce glycosphingolipid concentration in brain tissue whereby the concentration of GL-1 in the CSF is reduced. The concentration of GL-1 in the CSF can thus be an indicator of general lipid dysregulation and substrate accumulation in brain tissue of a subject, as well as a biomarker for assessing the reduction of glycosphingolipid concentration in brain tissue. The GL-1 levels in the CSF also reflect the levels of compound (e.g. Compound 2) in a dose-dependent manner.

As part of the methods disclosed herein, the glycosphingolipid concentration in brain tissue of the subject is reduced, as, for instance, observed by the concentration of GL-1 in the CSF of the subject being reduced by at least about 30% (i.e. after commencing treatment). In embodiments, the concentration of GL-1 in the CSF of the subject is reduced by at least about 35%, 40%, 45%, 50%, 55%, 60%, 65%, or 70% relative to pre-treatment levels (baseline). The greater the degree of GL-1 reduction in the CSF, the greater the impact there may be on modulating the glycosphingolipid environment in the subject's brain tissue. Achieving a reduction in GL-1 concentration in CSF of at least about 30% may thus require prolonged exposure to the quinuclidine compound, for instance as part of a long-term treatment plan that may be continued indefinitely. Thus, it is contemplated that the present methods will involve the administration of the quinuclidine compound over a period of at least about 1 week, e.g. at least about 2, 3, or 4 weeks, or at least about 1, 2, 3, 4, 6, or 12 months. In embodiments, the treatment continues beyond 12 months, e.g. indefinitely.

In embodiments, the therapeutic methods result in the aforementioned reduction in GL-1 concentration in the CSF within about 3 months of commencing treatment, e.g. within about 2 months, 1 month, 3 weeks, 2 weeks, or 1 week of commencing treatment.

It will be appreciated that, as substrate accumulation pathways may continue to predominate in patients, it is desirable to maintain a level of exposure of the quinuclidine compounds as described herein (e.g. Compound 2) in the CNS, particularly the brain tissue, as may be verified through monitoring CSF levels. The present disclosure therefore contemplates that the reduction in GL-1 concentration in the specified time period is achieved with regular dosing (e.g. daily, every other day, weekly, etc.) of the quinuclidine compound in order to maintain its exposure, e.g. at a steady state. The concentration of the quinuclidine compound as described herein (e.g. Compound 2) that is maintained in the CSF of the subject may be, e.g., at least about 0.1 ng/ml, for example at least about 0.5, 1, 1.5, 2, 3, 4, 5, 8, or 10 ng/ml. For example, the steady state level of the quinuclidine compound (e.g. Compound 2) that is maintained in the CSF of the subject may be, e.g., between about 0.1 and about 15 ng/ml, for example between about 1 and 10 ng/ml, or between about 2 and 8 ng/ml.

### Administration of the Quinuclidine Compound

The present inventors have found that a quinuclidine compound as described herein, in particular (S)-quinuclidin-3-yl (2-(2-(4-fluorophenyl)thiazol-4-yl)propan-2-yl)carbamate (Compound 2), has favorable safety and tolerability in human patients when administered orally. The prevailing pharmacodynamic properties of this compound, and other quinuclidine compounds described herein, that have been found by the inventors mean that such compounds may be administered as part of a regular dosage regimen and over a prolonged period of time. Furthermore, a quinuclidine compound as described herein (in particular, Compound 2), when administered orally to human patients, has been found by the inventors to have its exposure in plasma and CSF increased in a dose-proportional manner, and GL-1 levels in CSF were also found to decrease in a dose-dependent manner.

Accordingly, in embodiments the quinuclidine compound as described herein (e.g. Compound 2) is administered to the subject daily as part of the treatment methods described herein. Given the favorable safety and tolerability properties associated with quinuclidine compounds (e.g. Compound 2) that have been discovered by the inventors, a wide range of daily doses may be used in the methods of the disclosure, depending on severity of disease and, for instance, the degree of abnormality of the prevailing glycosphingolipid environment in the patient's brain tissue (as, for instance, determined by measurement of GL-1 levels in the patient's CSF). These doses could be determined by the skilled practitioner on the basis of the disclosure herein.

In embodiments, the quinuclidine compound (e.g. Compound 2) is administered daily. In other embodiments, the quinuclidine compound (e.g. Compound 2) is administered more than once daily, e.g. twice daily. In yet other embodiments, the quinuclidine compound (e.g. Compound 2) is administered less than once daily, e.g. every other day, every third day, or weekly.

In embodiments, the quinuclidine compound (e.g. Compound 2) is administered to the subject in a dose of about 0.5 mg to about 30 mg (or an equivalent amount of the prodrug or pharmaceutically acceptable salt thereof). For example, the quinuclidine compound (e.g. Compound 2) may be administered in a dose of from about 2 mg to about 20 mg, or from about 2 mg to about 10 mg, or from about 4 mg to about 8 mg, or from about 10 to about 20 mg, or from about 4 mg to about 15 mg. In other embodiments, the quinuclidine compound (e.g. Compound 2) is administered in a dose of from about 4 mg to about 18 mg, from about 8 mg to about 16 mg, or from about 10 mg to about 15 mg, e.g. a dose selected from about 4 mg, about 8 mg, and about 15 mg. In one embodiment, Compound 2 is administered at a daily dose of about 15 mg (or an equivalent amount of the prodrug or pharmaceutically acceptable salt thereof).

In embodiments, the administration of the quinuclidine compound as described herein (e.g. Compound 2) is via a non-parenteral route, e.g. via the oral route.

In one embodiment, a malate acid addition salt of (S)-quinuclidin-3-yl (2-(2-(4-fluorophenyl)thiazol-4-yl)propan-2-yl)carbamate (e.g. a crystalline malate salt thereof) is used in the methods of the disclosure, and is orally administered at a daily dose of from about 5 to about 20 mg, e.g. about 15 mg (measured as the equivalent amount of free base).

### Methods for Assessing Levels of Compound in CSF and Methods for Assessing the Effectiveness of Treatments

The present inventors have found that monitoring GL-1 concentration changes in the CSF of a subject who is administered a quinuclidine compound as defined herein (e.g. Compound 2) is a convenient means for assessing the exposure of the compound within the CNS; it may also represent a convenient means for assessing whether the treatment for Parkinson's disease (PD) or dementia with Lewy Bodies (DLB) is effective, for instance, in modulating the prevailing glycosphingolipid environment in brain tissue of the subject in order to achieve a positive therapeutic or physiological outcome.

Thus, in a further aspect, the invention provides a method of assessing the exposure of a quinuclidine compound within the CNS (e.g. within the CSF) of a human subject who has been administered a quinuclidine compound as defined herein (e.g. Compound 2); the method comprising determining the concentration of glucosylceramide (GL-1) in the cerebrospinal fluid (CSF) of the subject: i) prior to administration of the compound; and ii) after administration of the compound; and determining whether, and to what extent, there is a reduction in the concentration of GL-1 in the CSF of the subject.

In a yet further aspect, the invention provides a method of assessing the effectiveness of a treatment for PD or DLB in a human subject; the method comprising determining the concentration of glucosylceramide (GL-1) in the cerebrospinal fluid (CSF) of the subject: i) prior to commencement of the treatment; and ii) after commencement of the treatment; and determining whether, and to what extent, there is a reduction in the concentration of GL-1 in the CSF of the subject as a result of the treatment. In embodiments, the treatment for PD or DLB is by administration of a glucosylceramide synthase (GCS) inhibitor and, in particular, a quinuclidine compound as defined herein. In one embodiment the treatment for PD or DLB is oral administration of Compound 2 as defined herein.

The subject who is administered the compound or who receives the treatment may be a subject as defined herein in connection with the aforementioned therapeutic methods.

The concentration of GL-1 in the CSF of the subject may be determined using methods known to the skilled person. Typically, CSF is obtained by lumbar puncture and the sample may be used directly or purified, e.g. by column chromatography, to enrich the GL-1 fraction and/or to remove components which would otherwise interfere with the assay. The GL-1 in the CSF sample may be measured directly or, alternatively, it may be converted into another compound (e.g. by deacetylation or derivatisation with a fluorescent moiety) before measurement.

Methods for measuring glycosphingolipids such as GL-1 in clinical samples include LC-MS/MS methods (Zheng et al., Mol Gen Metabol Rep 2016; 8: 77-79; and Ji Ji et al., Bioanalysis 2015; 7(12): 1483-1496). One suitable method for the direct quantification of GL-1 in human CSF, e.g. for use in the present methods, is described in Xu et al. (J Lipid Res 2019; 60: 200-211). Xu et al. employ a LC/ESI/DMS/MS/MS method having quantitative performance for detection of GL-1 in human CSF at nM concentrations. Another option for quantifying GL-1 levels in CSF relies on hydrolysing the GL-1 in the CSF sample (which has low residual lyso-GL1 levels) and detecting the amount of lyso-GL1 which is formed using well-known methods of detecting lyso-GL1 in biological fluids at nM concentrations. By way of example, Taketomi et al. (J Biochem 1996; 120: 573-579) describe a microwave-mediated saponification method which can prepare lyso-compounds from the corresponding glycosphingolipids (including GL-1) at high yield.

### Compounds

Achieving a reduction in glycosphingolipid concentration in brain tissue through the methods disclosed herein requires exposure of the CNS to the pharmacological compound. The quinuclidine compounds described herein are active as inhibitors of the enzyme glucosylceramide synthase (“GCS”) and they have appropriate characteristics to penetrate the blood-brain barrier. They are, therefore, referred to as “brain-penetrant” quinuclidine compounds, and they may conveniently be administered via non-parenteral routes, for example orally. An example of an especially useful brain-penetrant quinuclidine compound according to the present disclosure is (S)-quinuclidin-3-yl (2-(2-(4-fluorophenyl)thiazol-4-yl)propan-2-yl)carbamate (Compound 2).

The quinuclidine compounds for use according to the present invention are compounds of formula (I),

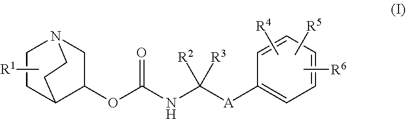

or a pharmaceutically acceptable salt or prodrug thereof, wherein:
R¹ is selected from hydrogen;
   halogen, cyano, nitro, hydroxy, thio, or amino, C₁₋₆-alkyl, C₂₋₆-alkenyl, C₂₋₆-alkynyl, C₁₋₆-alkyloxy, C₂₋₆-alkenyloxy, or C₂₋₆-alkynyloxy, wherein said alkyl, alkenyl, alkynyl, alkyloxy, alkenyloxy, or alkynyloxy is optionally substituted with one or more (e.g. 1, 2, or 3) groups independently selected from halogen, cyano, nitro, hydroxy, thio, or amino;
R² and R³ are each independently selected from C₁₋₃-alkyl, optionally substituted by one or more (e.g. 1, 2, or 3) halogens; or
R² and R³ together form a cyclopropyl or cyclobutyl group, optionally substituted by one or more (e.g. 1 or 2) halogens;
R⁴, R⁵, and R⁶ are each independently selected from hydrogen, halogen, nitro, hydroxy, thio, amino, C₁₋₆-alkyl, and C₁₋₆-alkyloxy, wherein said alkyl or alkyloxy is optionally substituted by one or more (e.g. 1, 2, or 3) groups selected from halogen, hydroxy, cyano, and C₁₋₆-alkyloxy; and
A is a 5- or 6-membered aryl or heteroaryl group, optionally substituted with 1, 2, or 3 groups independently selected from halogen, hydroxy, thio, amino, nitro, C₁₋₆-alkyloxy, and C₁₋₆-alkyl.

In one embodiment, R¹ is hydrogen; a halogen; or a C₁₋₄-alkyl or C₁₋₄-alkyloxy group, optionally substituted by one or two groups selected independently from a halogen; and a cyano, nitro, hydroxy, thio, or amino group. In another embodiment, R¹ is hydrogen; fluorine; or a methyl or ethyl group optionally substituted by a halogen, or a hydroxy, thio, or amino group. In a further embodiment, R¹ is hydrogen; or a methyl group optionally substituted by one or more (e.g. 1, 2, or 3) halogens. In a yet further embodiment, R¹ is hydrogen. In one embodiment, R¹ is not attached to the nitrogen atom of the quinuclidine moiety.

In one embodiment, R² and R³ are each independently selected from C₁₋₃-alkyl groups, optionally substituted with one or more halogens. In another embodiment, R² and R³ are each independently selected from methyl and ethyl groups, optionally substituted with one or more fluorine atoms. In a further embodiment, R² and R³ are each methyl, optionally substituted with one to three fluorine atoms. In a yet further embodiment, either R² and R³ are both methyl groups, or R² and R³ together form a cyclopropyl group. In a still further embodiment, R² and R³ are both methyl groups.

In one embodiment, R⁶ is hydrogen. In another embodiment, R⁵ and R⁶ are both hydrogen. In another embodiment at least one of R⁴, R⁵, and R⁶ is not hydrogen. In a further embodiment, R⁴ is selected from a halogen; and a C₁₋₃-alkyl or C₁₋₃-alkyloxy group, optionally substituted by one or more groups selected from a halogen; and a cyano or C₁₋₃-alkyloxy group. In another embodiment, R⁴ is selected from a halogen; and a C₁₋₃-alkyl or C₁₋₃-alkyloxy group, optionally substituted by one or more groups selected from a halogen; and a C₁₋₃-alkyloxy group. In a yet further embodiment, R⁴ is selected from fluorine; and a C₁₋₃-alkyloxy group, optionally substituted by one or more groups selected from a halogen; and a cyano or C₁₋₃-alkyloxy group. In a still further embodiment, R⁴ is selected from fluorine; and a C₁₋₃-alkyloxy group, optionally substituted by one or more groups selected from a halogen; and a cyano or C₁₋₃-alkyloxy group; and R⁵ and R⁶ are both hydrogen. For example, where R⁵ and R⁶ are both hydrogen, R⁴ may be fluorine or a 2-methoxyethoxy group, e.g. fluorine.

Where all of R⁴, R⁵, and R⁶ are other than hydrogen, these three groups may be attached to the benzene ring, for example, at positions 2, 4, and 6 (relative to the group A being attached to position 1). Where only one of R⁴, R⁵, and R⁶ is hydrogen, the other two groups may be attached to the benzene ring, for example, at positions 2 and 3, positions 3 and 4, or positions 3 and 5, e.g. at positions 3 and 5 (relative to the group A being attached to position 1). Where two of R⁴, R⁵, and R⁶ are hydrogen, the other group may be attached to the benzene ring at position 2, 3, or 4, e.g. at position 4 (i.e. at the position para to the group A). In one embodiment, R⁴ is in a position on the benzene ring para to the group A.

In one embodiment, A is a 6-membered aryl group or a 5-membered heteroaryl group. Non-limiting examples of 6-membered aryl groups and 5-membered heteroaryl groups include phenyl, furyl, thienyl, thiazolyl, pyrazolyl, isothiazolyl, oxazolyl, isoxazolyl, pyrrolyl, triazolyl, imidazolyl, oxadiazolyl, and thiadiazolyl. In one embodiment, the 6-membered aryl group or 5-membered heteroaryl group is selected from phenyl, thienyl, thiazolyl, pyrrolyl, and imidazolyl. In another embodiment, the 6-membered aryl group or 5-membered heteroaryl group is selected from phenyl and thiazolyl.

In one embodiment, A is phenyl, optionally substituted with 1, 2, or 3 groups independently selected from a halogen; and a hydroxy, thio, amino, nitro, oxy, or methyl group. In another embodiment, A is phenyl, optionally substituted with 1 or 2 halogens. In a further embodiment, A is phenyl, optionally substituted with a halogen, e.g. fluorine. In a yet further embodiment, A is an unsubstituted phenyl group.

Where A is 6-membered aryl or heteroaryl, the attached groups —C(R²R³)— and —(C₆H₂R⁴R⁵R⁶) may be in a 1,2- or 1,3- or 1,4-relationship, i.e. ortho, meta, or para to each other. In one embodiment, the attached groups —C(R²R³)— and —(C₆H₂R⁴R⁵R⁶) are in a 1,3-relationship. In another embodiment, the attached groups are in a 1,4-relationship.

In one embodiment, A is a 5-membered heteroaryl group which contains 1, 2, or 3 heteroatoms selected from N, O, and S. In another embodiment, A is a 5-membered heteroaryl group which contains 1 or 2 heteroatoms selected from N and S. In a further embodiment, A is a 5-membered heteroaryl group which contains 2 heteroatoms selected from N and S. In a yet further embodiment, A is a 5-membered heteroaryl group which contains 2 heteroatoms wherein one heteroatom is N and the other heteroatom is S. In a still further embodiment, A is a thiazolyl group.

Where A is a 5-membered heteroaryl group, at least one of the attached groups —C(R²R³)— and —(C₆H₂R⁴R⁵R⁶) may be bonded directly to a carbon atom of the heteroaryl group. In one embodiment, both of the attached groups —C(R²R³)— and —(C₆H₂R⁴R⁵R⁶) are bonded directly to a carbon atom of the heteroaryl group. In one embodiment, the attached groups —C(R²R³)— and —(C₆H₂R⁴R⁵R⁶) are in a 1,3-relationship to each other, e.g. they are bonded directly to carbon atoms of the heteroaryl group which are separated by a single intervening atom, e.g. heteroatom. In the embodiment where A is a thiazolyl group, the attached groups —C(R²R³)— and —(C₆H₂R⁴R⁵R⁶) may be bonded directly at the 4- and 2-positions, respectively.

Thus, in one embodiment the quinuclidine compound is a compound of formula (II),

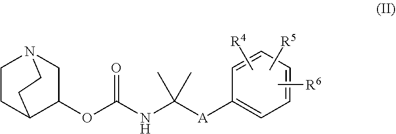

or a pharmaceutically acceptable salt or prodrug thereof, wherein R⁴, R⁵, R⁶, and A are as defined herein.

In another embodiment, the quinuclidine compound is a compound of formula (III),

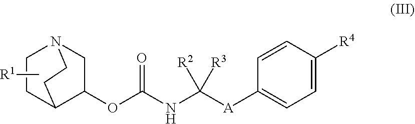

or a pharmaceutically acceptable salt or prodrug thereof, wherein R¹ to R⁴, and A are as defined herein.

In another embodiment, the quinuclidine compound is a compound of formula (IV),

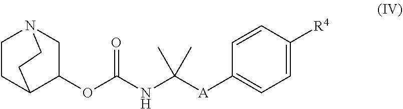

or a pharmaceutically acceptable salt or prodrug thereof, wherein R⁴ and A are as defined herein.

In one embodiment, R⁴ is a halogen, e.g. fluorine. Accordingly, the quinuclidine compound may be a compound of formula (V),

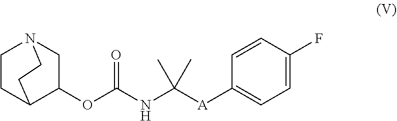

or a pharmaceutically acceptable salt or prodrug thereof, wherein A is as defined herein.

In another embodiment, the quinuclidine compound is a compound of formula (VI),

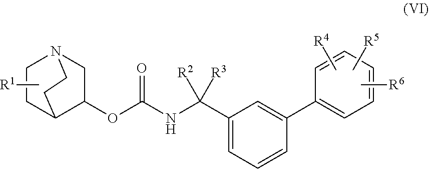

or a pharmaceutically acceptable salt or prodrug thereof, wherein R¹ to R⁶ are as defined herein.

In another embodiment, the quinuclidine compound is a compound of formula (VII),

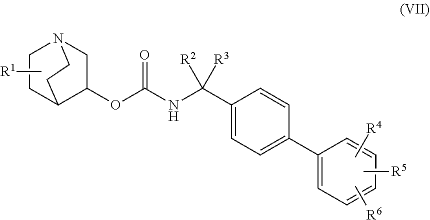

or a pharmaceutically acceptable salt or prodrug thereof, wherein R¹ to R⁶ are as defined herein.

In another embodiment, the quinuclidine compound is a compound of formula (VIII),

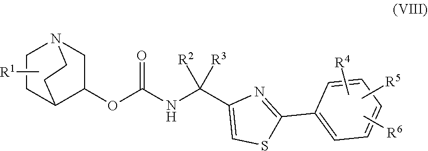

or a pharmaceutically acceptable salt or prodrug thereof, wherein R¹ to R⁶ are as defined herein.

In another embodiment, the quinuclidine compound is a compound of formula (IX),

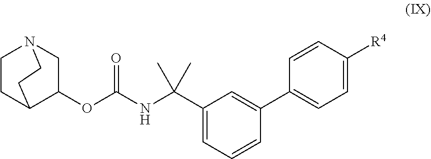

or a pharmaceutically acceptable salt or prodrug thereof, wherein R⁴ is as defined herein.

In one embodiment, R⁴ is a halogen, e.g. fluorine. Accordingly, the quinuclidine compound may be a compound of formula (X),

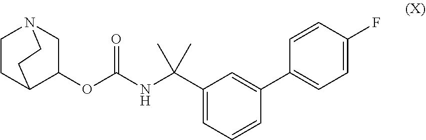 [SMILES: CC(C)(NC(=O)OC1CN2CCC1CC2)c1cccc(-c2ccc(F)cc2)c1]

or a pharmaceutically acceptable salt or prodrug thereof.

In another embodiment, the quinuclidine compound is a compound of formula (XI),

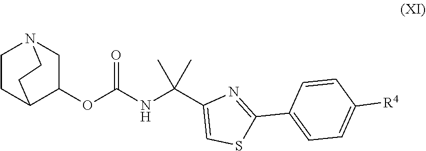

or a pharmaceutically acceptable salt or prodrug thereof, wherein R⁴ is as defined herein.

In one embodiment, R⁴ is a halogen, e.g. fluorine. Accordingly, the quinuclidine compound may be a compound of formula (XII),

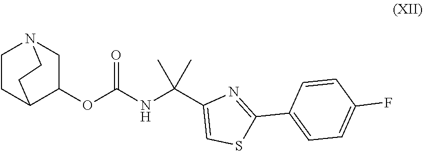 [SMILES: CC(C)(NC(=O)OC1CN2CCC1CC2)c1csc(-c2ccc(F)cc2)n1]

or a pharmaceutically acceptable salt or prodrug thereof.

In one embodiment, the quinuclidine compound is selected from the group consisting of Compound 1 to Compound 23:

| Compound | |
|---|---|
| No. | Compound |
| 1 | Quinuclidin-3-yl (2-(4′-fluoro-[1,1′-biphenyl]-3-yl)propan- |
| | 2-yl)carbamate |
| 2 | (S)-quinuclidin-3-yl (2-(2-(4-fluorophenyl)thiazol-4- |
| | yl)propan-2-yl)carbamate |
| 3 | (S)-quinuclidin-3-yl (2-(4′-(2-methoxyethoxy)-[1,1′- |
| | biphenyl]-4-yl)propan-2-yl)carbamate |
| 4 | 1-azabicyclo[2.2.2]oct-3-yl [2-(biphenyl-3-yl)propan-2- |
| | yl]carbamate |
| 5 | (S)-quinuclidin-3-yl 2-(biphenyl-4-yl)propan-2-ylcarbamate |
| 6 | Quinuclidin-3-yl 1-(biphenyl-4-yl)cyclopropylcarbamate |
| 7 | (S)-quinuclidin-3-yl 1-(4′-fluorobiphenyl-4- |
| | yl)cyclopropylcarbamate |
| 8 | (S)-1-azabicyclo[2.2.2]oct-3-yl [1-(2′,4′-difluorobiphenyl-4- |
| | yl)cyclopropyl]carbamate |
| 9 | 1-azabicyclo[2.2.2]oct-3-yl [1-(4′-methoxybiphenyl-4- |
| | yl)cyclopropyl]carbamate |
| 10 | Quinuclidin-3-yl 2-(5-(4-fluorophenyl)thiophen-3- |
| | yl)propan-2-ylcarbamate |
| 11 | (S)-quinuclidin-3-yl 2-(3-(4-fluorophenyl)isothiazol-5- |
| | yl)propan-2-ylcarbamate |
| 12 | (S)-quinuclidin-3-yl 2-(4-(4-fluorophenyl)thiazol-2- |
| | yl)propan-2-ylcarbamate |
| 13 | Quinuclidin-3-yl (2-(4′-(2-methoxyethoxy)-[1,1′-biphenyl]- |
| | 4-yl)propan-2-yl)carbamate |
| 14 | (S)-quinuclidin-3-yl (2-(3′-(2-methoxyethoxy)-[1,1′- |
| | biphenyl]-4-yl)propan-2-yl)carbamate |
| 15 | Quinuclidin-3-yl (2-(4′-(2-methoxyethoxy)-[1,1′-biphenyl]- |
| | 3-yl)propan-2-yl)carbamate |
| 16 | Quinuclidin-3-yl (2-(4′-(3-methoxypropoxy)-[1,1′- |
| | biphenyl]-4-yl)propan-2-yl)carbamate |
| 17 | Quinuclidin-3-yl (2-(4′-(hydroxymethyl)-[1,1′-biphenyl]- |
| | 4-yl)propan-2-yl)carbamate |
| 18 | Quinuclidin-3-yl (2-(4′-(2-hydroxyethyl)-[1,1′-biphenyl]- |
| | 4-yl)propan-2-yl)carbamate |
| 19 | Quinuclidin-3-yl (2-(2-(4-(3- |
| | methoxypropoxy)phenyl)thiazol-4-yl)propan-2- |
| | yl)carbamate |
| 20 | Quinuclidin-3-yl (2-(2-(4-(2-methoxyethoxy)phenyl)thiazol- |
| | 4-yl)propan-2-yl)carbamate |
| 21 | Quinuclidin-3-yl 2-(5-(4-(2-methoxyethoxy)phenyl)pyridin- |
| | 2-yl)propan-2-ylcarbamate |
| 22 | Quinuclidin-3-yl (2-(4′-(3-cyanopropoxy)-[1,1′-biphenyl]-4- |
| | yl)propan-2-yl)carbamate |
| 23 | Quinuclidin-3-yl (2-(4′-(cyanomethoxy)-[1,1′-biphenyl]-4- |
| | yl)propan-2-yl)carbamate |

and the pharmaceutically acceptable salts and prodrugs thereof.

In one embodiment, the quinuclidine compound is selected from Compound 1, Compound 2, and Compound 3, and the pharmaceutically acceptable salts and prodrugs thereof. In another embodiment, the quinuclidine compound is selected from Compound 1 and Compound 3, and the pharmaceutically acceptable salts and prodrugs thereof. In another embodiment, the quinuclidine compound is Compound 1, or a pharmaceutically acceptable salt or prodrug thereof. In another embodiment, the quinuclidine compound is Compound 2, or a pharmaceutically acceptable salt or prodrug thereof. In another embodiment, the quinuclidine compound is Compound 3, or a pharmaceutically acceptable salt or prodrug thereof.

In another embodiment, the quinuclidine compound is selected from Compound 1, Compound 2, and Compound 3. In one embodiment, the quinuclidine compound is Compound 1. In another embodiment, the quinuclidine compound is Compound 2. In another embodiment, the quinuclidine compound is Compound 3.

### Salts

Presently disclosed compounds that are basic in nature are generally capable of forming a wide variety of different salts with various inorganic and/or organic acids. Although such salts are generally pharmaceutically acceptable for administration to animals and humans, it is often desirable in practice to initially isolate a compound from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the base compounds can be readily prepared using conventional techniques, e.g. by treating the base compound with a substantially equivalent amount of the chosen mineral or organic acid in an aqueous solvent medium or in a suitable organic solvent such as, for example, methanol or ethanol. Upon careful evaporation of the solvent, the desired solid salt is obtained. Presently disclosed compounds that are positively charged, e.g. containing a quaternary ammonium, may also form salts with the anionic component of various inorganic and/or organic acids. Acids which can be used to prepare pharmaceutically acceptable salts of quinuclidine compounds are those which can form non-toxic acid addition salts, e.g. salts containing pharmacologically acceptable anions, such as chloride, bromide, iodide, nitrate, sulfate or bisulfate, phosphate or acid phosphate, acetate, lactate, citrate or acid citrate, tartrate or bitartrate, succinate, malate, maleate, fumarate, gluconate, saccharate, benzoate, methanesulfonate, and pamoate [i.e. 1,1′-methylene-bis-(2-hydroxy-3-naphthoate)] salts.

Presently disclosed compounds that are acidic in nature, e.g. compounds containing a tetrazole moiety, are generally capable of forming a wide variety of different salts with various inorganic and/or organic bases. Although such salts are generally pharmaceutically acceptable for administration to animals and humans, it is often desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g. by treating the corresponding acidic compounds with an aqueous solution containing the desired pharmacologically acceptable cations, and then evaporating the resulting solution to dryness, e.g. under reduced pressure. Alternatively, they also can be prepared by mixing lower alkanolic solutions of the acidic compounds and the desired alkali metal alkoxide together, and then evaporating the resulting solution to dryness in the same manner as before. In either case, stoichiometric quantities of reagents may be employed in order to ensure completeness of reaction and maximum product yields of the desired solid salt.

Bases which can be used to prepare the pharmaceutically acceptable base addition salts of quinuclidine compounds are those which can form non-toxic base addition salts, e.g. salts containing pharmacologically acceptable cations, such as, alkali metal cations (e.g. potassium and sodium), alkaline earth metal cations (e.g. calcium and magnesium), ammonium or other water-soluble amine addition salts such as N-methylglucamine (meglumine), lower alkanolammonium, and other such bases of organic amines.

In one embodiment, the pharmaceutically acceptable salt is a succinate salt. In another embodiment, the pharmaceutically acceptable salt is a hydroxysuccinate salt, e.g. an (S)-2-hydroxysuccinate salt. In another embodiment, the pharmaceutically acceptable salt is a hydrochloride salt (i.e. a salt with HCl). In another embodiment, the pharmaceutically acceptable salt is a malate salt. In one embodiment, the quinuclidine compound is the malate salt of Compound 2.

### Prodrugs

The pharmaceutically acceptable prodrugs disclosed herein are derivatives of quinuclidine compounds which can be converted in vivo into the quinuclidine compounds described herein. The prodrugs, which may themselves have some activity, become pharmaceutically active in vivo when they undergo, for example, solvolysis under physiological conditions or enzymatic degradation. Methods for preparing prodrugs of compounds as described herein would be apparent to one of skill in the art based on the present disclosure.

In one embodiment, the carbamate moiety of the quinuclidine compound is modified. For example, the carbamate moiety of the quinuclidine compound may be modified by the addition of water and/or one or two aliphatic alcohols. In this case, the carbon-oxygen double bond of the carbamate moiety adopts what could be considered a hemiacetal or acetal functionality. In one embodiment, the carbamate moiety of the quinuclidine compound may be modified by the addition of an aliphatic diol such as 1,2-ethanediol.

In one embodiment, one or more of the hydroxy, thio, or amino groups on the quinuclidine compound are modified. For example, one or more of the hydroxy, thio, and/or amino groups on the quinuclidine compound may be modified to form acid derivatives, e.g. esters, thioesters (or thiolesters), and/or amides. The acid derivatives can be formed, for example, by reacting a quinuclidine compound which comprises one or more hydroxy, thio, or amino groups with an acetylating agent. Examples of acetylating agents include anhydrides such as acetic anhydride, acid chlorides such as benzyl chloride, and dicarbonates such as di-tert-butyl dicarbonate.

### Stereochemistry

Stereoisomers (e.g. cis and trans isomers) and all optical isomers of a presently disclosed compound (e.g. R- and S-enantiomers), as well as racemic, diastereomeric, and other mixtures of such isomers are within the scope of the present disclosure.

In one embodiment, the quinuclidin-3-yl group of a quinuclidine compound as defined herein has the R-configuration. Accordingly, the quinuclidine compound may be selected from the group consisting of compounds of formulae (Ia) to (XIIa):

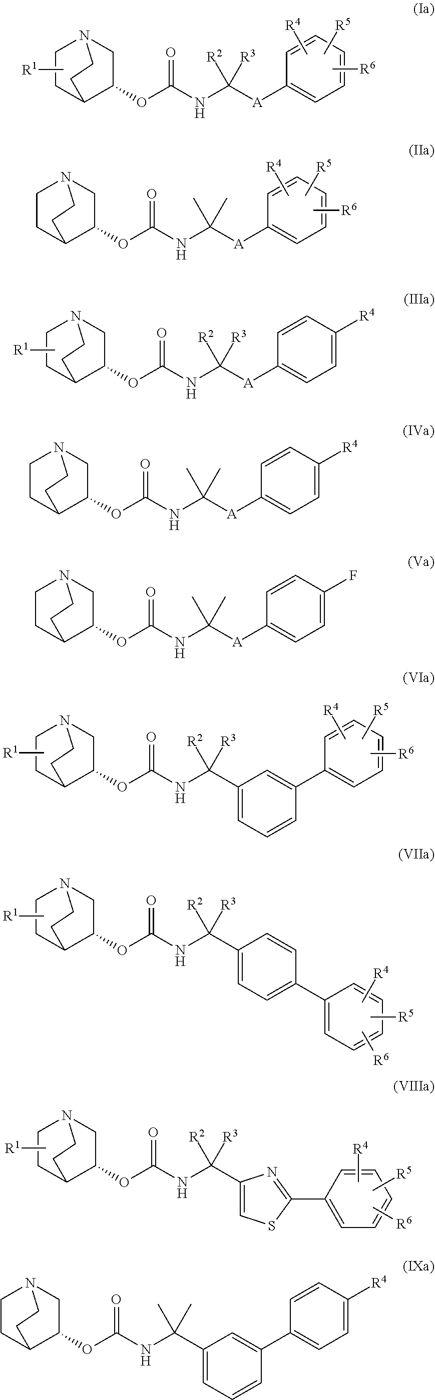

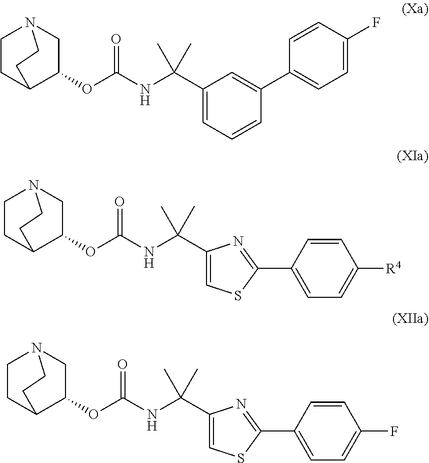

and the pharmaceutically acceptable salts and prodrugs thereof.

In another embodiment, the quinuclidin-3-yl group of the quinuclidine compound as defined herein has the S-configuration. Accordingly, the quinuclidine compound may be selected from the group consisting of compounds of formulae (Ib) to (XIIb):

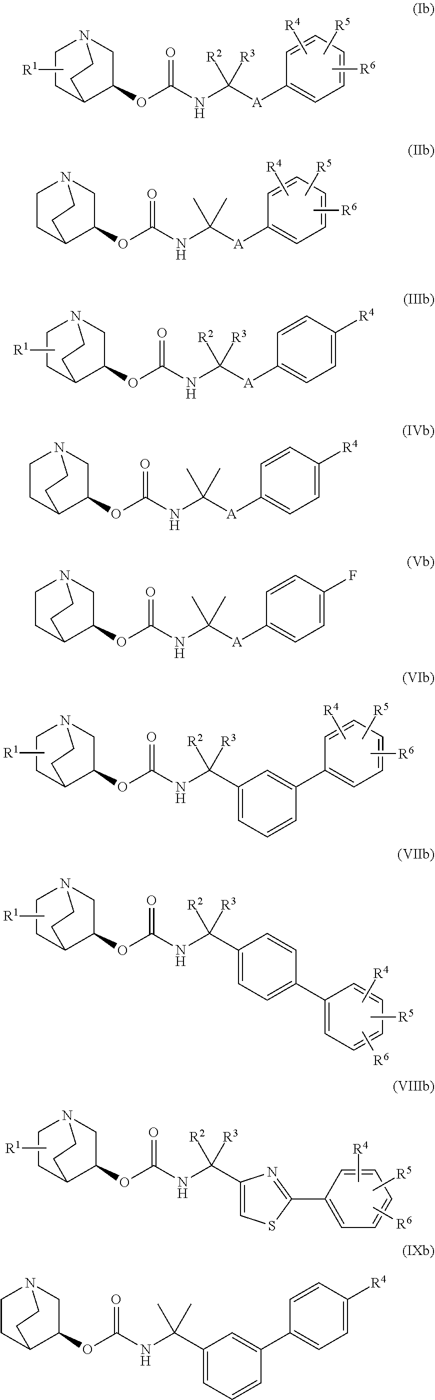

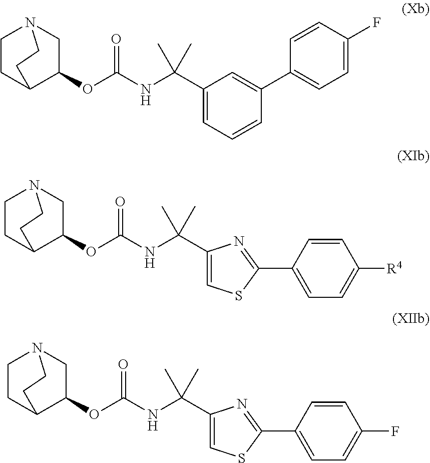

and the pharmaceutically acceptable salts and prodrugs thereof.

In one embodiment the quinuclidine compound is a compound of formula (Xb), or a pharmaceutically acceptable salt or prodrug thereof. In another embodiment the quinuclidine compound is a compound of formula (XIIb), or a pharmaceutically acceptable salt or prodrug thereof.

In one embodiment, the quinuclidin-3-yl group of the quinuclidine compound as defined herein exists in a mixture of isomers having the R- and S-configurations. For example, the quinuclidine compound may be a mixture of compounds selected from the group consisting of compounds of formulae (Ia) and (Ib), (IIa) and (IIb), (IIIa) and (IIIb), (IVa) and (IVb), (Va) and (Vb), (VIa) and (VIb), (VIIa) and (VIIb), (VIIIa) and (VIIIb), (IXa) and (IXb), (Xa) and (Xb), (XIa) and (XIb), and (XIIa) and (XIIb), and the pharmaceutically acceptable salts and prodrugs thereof. In one embodiment the quinuclidine compound is present as a racemic mixture, e.g. the R- and S-isomers of the quinuclidin-3-yl group are present in about equal amounts. In another embodiment the quinuclidine compound is present as a mixture of isomers having the R- and S-configurations, wherein the R- and S-isomers are present in different amounts. In one embodiment the S-isomer is present in an enantiomeric excess of at least about 5%, 10%, 25%, 40%, 70%, 80%, 90%, 95%, 97%, 98%, or 99%, e.g. about 100%. In another embodiment, the R-isomer is present in an enantiomeric excess of at least about 5%, 10%, 25%, 40%, 70%, 80%, 90%, 95%, 97%, 98%, or 99%, e.g. about 100%.

Methods for preparing enantioenriched and/or enantiopure quinuclidine compounds would be apparent to the person of skill in the art based on the present disclosure.

The compounds presently disclosed can exist in several tautomeric forms, including the enol and imine form, and the keto and enamine form, and geometric isomers and mixtures thereof. Tautomers exist as mixtures of a tautomeric set in solution. In solid form, usually one tautomer predominates. Even though one tautomer may be described, all tautomers are within the scope of the present disclosure.

Atropisomers are also within the scope of the present disclosure. Atropisomers refer to compounds that can be separated into rotationally restricted isomers.

### Other Forms

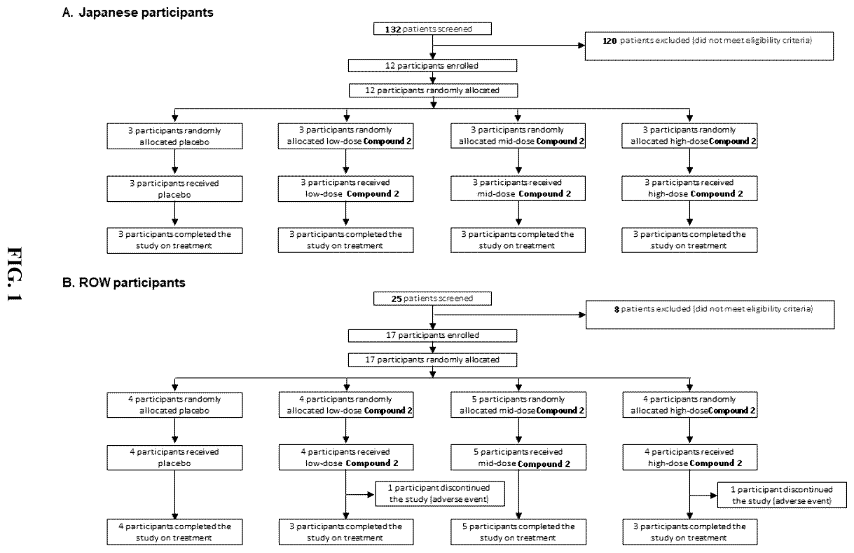

Pharmaceutically acceptable hydrates, solvates, polymorphs, etc., of the quinuclidine compounds described herein are within the scope of the present disclosure. Quinuclidine compounds as described herein may be in an amorphous form and/or in one or more crystalline forms. In an embodiment, a crystalline salt form of (S)-quinuclidin-3-yl (2-(2-(4-fluorophenyl)thiazol-4-yl)propan-2-yl)carbamate (Compound 2) may be used in the methods disclosed herein, such as the crystalline malate salt Form A as disclosed in, e.g., US 2016/0039805 (the content of which is hereby incorporated by reference in its entirety), with particular reference being made to paragraphs [0005] to [0010] and FIG. 1 of that document.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine, and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

### Pharmaceutical Compositions

The present disclosure also provides pharmaceutical compositions comprising at least one quinuclidine compound as described herein (e.g. Compound 2) and at least one pharmaceutically acceptable excipient, e.g. for use according to the methods disclosed herein. The pharmaceutically acceptable excipient can be any such excipient known in the art including those described in, for example, Remington's Pharmaceutical Sciences, Mack Publishing Co. (A. R. Gennaro edit. 1985). Pharmaceutical compositions of the compounds presently disclosed may be prepared by conventional means known in the art including, for example, mixing at least one presently disclosed compound with a pharmaceutically acceptable excipient.

Thus, in embodiments the quinuclidine compound as described herein (e.g. Compound 2) is included in a pharmaceutical dosage form together with a pharmaceutically acceptable excipient, wherein the dosage form is formulated to provide, when administered (e.g. when administered orally), an amount of said compound sufficient to reduce glycosphingolipid concentration in brain tissue of the human subject, whereby the concentration of GL-1 in the CSF of the subject is reduced (e.g. by at least about 30%). In one embodiment, said subject has Parkinson's disease (PD). In another embodiment, said subject has dementia with Lewy Bodies (DLB).

A pharmaceutical composition or dosage form for use in the methods of the invention can include an agent and another carrier, e.g. compound or composition, inert or active, such as a detectable agent, label, adjuvant, diluent, binder, stabilizer, buffers, salts, lipophilic solvents, preservative, adjuvant, or the like. Carriers also include pharmaceutical excipients and additives, for example, proteins, peptides, amino acids, lipids, and carbohydrates (e.g. sugars, including monosaccharides, di-, tri-, tetra-, and oligosaccharides; derivatized sugars such as alditols, aldonic acids, esterified sugars, and the like; and polysaccharides or sugar polymers), which can be present singly or in combination, comprising alone or in combination 1 to 99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Examples of carbohydrates include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), and myoinositol.

Carriers which may be used include a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g. cyclodextrins, such as 2-hydroxypropyl-p-cyclodextrin), polyethylene glycols, flavoring agents, antimicrobial agents, sweeteners, antioxidants, antistatic agents, surfactants (e.g. polysorbates such as “TWEEN 20” and “TWEEN 80”), lipids (e.g. phospholipids, fatty acids), steroids (e.g. cholesterol), and chelating agents (e.g. EDTA).

The present disclosure also contemplates pharmaceutical compositions, and kits comprising said compositions, which contain at least one quinuclidine compound as described herein (e.g. Compound 2) and at least one further pharmaceutically-active agent, e.g. for use in the methods described herein. These pharmaceutical compositions and kits may be adapted to allow simultaneous, subsequent, and/or separate administration of the quinuclidine compound and the further active agent. For example, the quinuclidine compound and the further active agent may be formulated in separate dosage forms, e.g. in separate tablets, capsules, lyophilisates, or liquids, or they may be formulated in the same dosage form, e.g. in the same tablet, capsule, lyophilisate, or liquid. Where the quinuclidine compound and the further active agent are formulated in the same dosage form, the quinuclidine compound and the further active agent may be present substantially in admixture, e.g. within the core of a tablet, or they may be present substantially in discrete regions of the dosage form, e.g. in separate layers of the same tablet. In embodiments, the further active agent is capable of treating or preventing Parkinson's disease or dementia with Lewy Bodies.

In embodiments, a pharmaceutical composition comprising: (i) a quinuclidine compound as described herein; (ii) a further active agent; and (iii) a pharmaceutically acceptable excipient, may be used in the methods described herein. In embodiments, the further active agent is an agent which is capable of treating or preventing Parkinson's disease or dementia with Lewy Bodies when administered orally to a subject.

Examples of further agents capable of treating Parkinson's disease include, for example, dopamine precursors (e.g. levodopa/carbidopa), dopamine agonists (e.g. ropinirole, bromocriptine, cabergoline, pergolide, pramipexole, and apomorphine), MAO-B inhibitors (e.g. rasagiline and selegiline), anticholinergics (e.g. artane, cogentin), adamantane derivatives, and acetylcholinesterase inhibitors such as tacrine, rivastigmine, galantamine, donepezil, and memantine.

Although specific embodiments of the present disclosure will now be described with reference to the preparations, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

### EXAMPLES

### Example 1: Preparation of Quinuclidine Compounds

The quinuclidine compounds disclosed herein may be prepared in accordance with published synthetic procedures, for example as described in WO 2016/145046. By way of example, a synthetic method is described below for the preparation of Compound 2 and a salt form thereof:

(S)-quinuclidin-3-yl 2-(2-(4-fluorophenyl)thiazol-4-yl)propan-2-ylcarbamate (Compound 2) To a stirred solution of 4-fluorothiobenzamide (8.94 g, 57.6 mmol) in ethanol (70 mL) was added ethyl 4-chloroacetoacetate (7.8 mL, 58 mmol). The reaction was heated at reflux for 4 hours, treated with an addition aliquot of ethyl 4-chloroacetoacetate (1.0 mL, 7.4 mmol), and refluxed for an additional 3.5 hours. The reaction was then concentrated and the residue was partitioned between ethyl acetate (200 mL) and aqueous NaHCO₃ (200 mL). The organic layer was combined with a back extract of the aqueous layer (ethyl acetate, 1×75 mL), dried (Na₂SO₄), and concentrated. The resulting amber oil was purified by flash chromatography using a hexane/ethyl acetate gradient to afford ethyl 2-(2-(4-fluorophenyl)thiazol-4-yl)acetate as a low melting, nearly colourless solid (13.58 g, 89%).

To a stirred solution of ethyl 2-(2-(4-fluorophenyl)thiazol-4-yl)acetate (6.28 g, 23.7 mmol) in DMF (50 mL) was added sodium hydride [60% dispersion in mineral oil] (2.84 g, 71.0 mmol). The frothy mixture was stirred for 15 minutes before cooling in an ice bath and adding iodomethane (4.4 mL, 71 mmol). The reaction was stirred overnight, allowing the cooling bath to slowly warm to room temperature. The mixture was then concentrated, and the residue partitioned between ethyl acetate (80 mL) and water (200 mL). The organic layer was washed with a second portion of water (1×200 mL), dried (Na₂SO₄), and concentrated.

The resulting amber oil was purified by flash chromatography using a hexane/ethyl acetate gradient to afford ethyl 2-(2-(4-fluorophenyl)thiazol-4-yl)-2-methylpropanoate as a colourless oil (4.57 g, 66%).

To a stirred solution of ethyl 2-(2-(4-fluorophenyl)thiazol-4-yl)-2-methylpropanoate (4.56 g, 15.5 mmol) in 1:1:1 THF/ethanol/water (45 mL) was added lithium hydroxide monohydrate (2.93 g, 69.8 mmol). The reaction was stirred overnight, concentrated and redissolved in water (175 mL). The solution was washed with ether (1×100 mL), acidified by the addition of 1.0 N HCl (80 mL), and extracted with ethyl acetate (2×70 mL). The combined extracts were dried (Na₂SO₄) and concentrated to afford 2-(2-(4-fluorophenyl)thiazol-4-yl)-2-methylpropanoic acid as a white solid (4.04 g, 98%). This material was used in the next step without purification.

To a stirred and cooled (0° C.) solution of 2-(2-(4-fluorophenyl)thiazol-4-yl)-2-methylpropanoic acid (4.02 g, 15.2 mmol) in THE (100 mL) was added trimethylamine (4.2 mL, 30 mmol) followed by isobutyl chloroformate (3.0 mL, 23 mmol). The reaction was stirred cold for another 1 hour before adding a solution of sodium azide (1.98 g, 30.5 mmol) in water (20 mL). The reaction was stirred overnight, allowing the cooling bath to slowly warm to room temperature. The mixture was then diluted with water (100 mL) and extracted with ethyl acetate (2×60 mL). The combined extracts were washed with aqueous NaHCO₃ (1×150 mL) and brine (1×100 mL), dried (Na₂SO₄), and concentrated. After coevaporating with toluene (2×50 mL), the resulting white solid was taken up in toluene (100 mL) and refluxed for 4 hours. (S)-3-quinuclidinol (3.87 g, 30.4 mmol) was then added and reflux was continued overnight. The reaction was concentrated, and the residue partitioned between ethyl acetate (100 mL) and aqueous NaHCO₃ (150 mL). The organic layer was washed with water (1×150 mL), dried (Na₂SO₄), and concentrated. The resulting off-white solid was purified by flash chromatography using a chloroform/methanol/ammonia gradient to afford the title compound as a white solid (4.34 g, 73%). ¹H NMR (400 MHz, CDCl₃) δ 7.96-7.88 (m, 2H), 7.16-7.04 (m, 3H), 5.55 (br s, 1H), 4.69-4.62 (m, 1H), 3.24-3.11 (m, 1H), 3.00-2.50 (m, 5H), 2.01-1.26 (m, 11H) ppm. ¹³C NMR (400 MHz, CDCl₃) δ 166.4, 165.1, 163.8 (d, J=250.3 Hz), 162.9, 155.0, 130.1 (d, J=3.3 Hz), 128.4 (d, J=8.5 Hz), 115.9 (d, J=22.3 Hz), 112.5, 71.2, 55.7, 54.2, 47.5, 46.5, 28.0, 25.5, 24.7, 19.6 ppm. Purity: 100% UPLCMS (210 nm & 254 nm); retention time 0.83 min; (M+1) 390.

Crystalline Forms of (S)-Quinuclidin-3-yl (2-(2-(4-fluorophenyl)thiazol-4-yl)propan-2-yl)carbamate Salts

Crystalline salts of (S)-Quinuclidin-3-yl (2-(2-(4-fluorophenyl)thiazol-4-yl)propan-2-yl)carbamate may be formed, for example, by dissolving the free base in IPA (140 ml) at room temperature and filtered. The filtrate is added into a 1 L r.b. flask which is equipped with an overhead stirrer and nitrogen in/outlet. L-malic acid (about 50 mmol) is dissolved in IPA (100+30 ml) at room temperature and filtered. The filtrate is added into the above 1 Liter flask. The resulting solution is stirred at room temperature (with or without seeding) under nitrogen for 4 to 24 hours. During this period of time crystals form. The product is collected by filtration and washed with a small amount of IPA (30 ml). The crystalline solid is dried in a vacuum oven at 55° C. for 72 hours to yield the desired malate salt.

Crystal forms of other salts, e.g. acid addition salts with succinic acid or HCl, may be prepared in an analogous manner.

### Example 2: Clinical Study of Compound 2 in Human Patients with PD

### Study Design and Participants

This clinical trial is a 3-year, phase 2, randomised, double-blinded, placebo-controlled, multicenter study of Compound 2 in participants with early-stage PD carrying a GBA mutation. The trial is divided into 2 consecutive parts: part 1 is a dose escalation study (described below) of safety, pharmacokinetics, and pharmacodynamics of 3 doses using 3 sequential cohorts; part 2 is an ongoing 2-arm study of efficacy and safety of Compound 2.

In part 1 of the study, which has been completed, Compound 2 dose was escalated in sequential cohorts; only when safety and tolerability were demonstrated after data review when all participants completed the first 4-week course of therapy, escalation to the next higher level in the next cohort could occur.

### Inclusion Criteria

Eligible participants were aged 18-80 years, had diagnosis of PD, as defined by ≥2 of the following signs: resting tremor, postural instability, akinesia/hypokinesia, and muscle rigidity; with PD symptoms for ≥2 years, were at stage 2 or less at baseline on the Hoehn and Yahr scale (Hoehn et al., Neurology 1967; 17: 427-42; Goetz et al., Mov Disord 2004; 19:1020-8), and were heterozygous carriers of a GBA mutation. A comprehensive list of screened GBA mutations is shown in Table 1, and included severe mutations, known to cause Gaucher Disease (GD) types 2 and 3, and other, non-severe, mutations, including mild GBA mutations that are present in patients with GD type 1.

For participants carrying known sequence variants associated with GBA-PD (e.g. E326K or T369M), history of rapid eye movement sleep behaviour disorder and/or co-occurrence with any mutation listed was required.

TABLE 1
List of common GBA mutations screened
to determine enrolment eligibility^{a}

| | Amino acid | cDNA nucleotide |
|---|---|---|
| Category | substitution | substitution |
| Severe GBA mutations | Frameshift | 122insC |
| | Frameshift | 203delC |
| | Frameshift | 329-333del CAGAA |
| | Frameshift | 330delA |
| | Frameshift | 493insA |
| | Frameshift | 500insT |
| | Frameshift | 532delC |
| | Frameshift | 534delT |
| | Frameshift | 595-596delCT |
| | Frameshift | 72delC |
| | Frameshift | 741delC |
| | Frameshift | 84G > GG |
| | Frameshift | 898delG |
| | Frameshift | 914C > del7 |
| | Frameshift | 953delT7 |
| | Frameshift | 1093-1094insG |
| | Frameshift | 1098A > AA8 |
| | Frameshift | 1122-1123insTG |
| | Frameshift | 1129delT |
| | Frameshift | 1147delG |
| | Frameshift | 119delC |
| | Frameshift | 1214GCdel8 |
| | Frameshift | 1263-1317del |
| | Frameshift | 1326insT |
| | Frameshift | 1447-1466del TGins |
| | A190T | 685G > A |
| | A190E | 686C > A |
| | A341T | 1138G > A |
| | A456P | 1483G > C |
| | C16S | 164G > C |
| | C342Y | 1142G > A |
| | D380A | 1256A > C |
| | D399N | 1312G > A |
| | D409H | 1342G > C |
| | D409V | 1343A > T |
| | E41K | 238G > A |
| | E233Stop | 814G > T |
| | E388Stop | 1279G > T |
| | F213I | 754T > A |
| | F251L | 870C > A |
| | G195E | 701G > A |
| | G195W | 700G > T |
| | G202R | 721G > A |
| | G325R | 1090G > A |
| | G355D | 1181G > A |
| | G389E | 1283G > A |
| | G390R | 1285G > A |
| | H255Q | 882T > G |
| | H311R | 1049A > G |
| | I119T | 473T > C |
| | I402F | 1321A > T |
| | K157Q | 586A > C |
| | K157N | 588G > T |
| | K198E | 710A > G |
| | K425E | 1390A > G |
| | L197F | 706C > T |
| | L444P | 1448G > C |
| | L444R | 1448T > G |
| | N392I | 1292A > T |
| | N462K | 1503C > G |
| | P178S | 649C > T |
| | P182L | 662C > T |
| | P266L | 915C > T |
| | P415R | 1361C > G |
| | Q73Stop | 334C > T |
| | Q169Stop | 622C > T |
| | Q350Stop | 1165C > T |
| | Q414Stop | 1357C > T |
| | Q414R | 1358A > G |
| | R47Stop | 256C > T |
| | R120Q | 476G > A |
| | R131C | 508C > T |
| | R131L | 509G > T |
| | R163Stop | 604C > T |
| | R257Q | 887G > A |
| | R257Stop | 886C > T |
| | R285H | 971G > A |
| | R353G | 1174C > G |
| | R359Stop | 1192C > T |
| | R463C | 1504C > T |
| | R463Q | 1505G > A |
| | S107L | 437C > T |
| | S173Stop | 635C > G |
| | S237P | 826T > C |
| | S364R | 1207A > C |
| | T231R | 809C > G |
| | T491I | 1589C > T |
| | V394L | 1297G > T |
| | V398L | 1309G > C |
| | V398F | 1309G > T |
| | W179Stop | 653G > A |
| | W(−)4Stop | 108G > A |
| | W378Stop | 1250G > A |
| | Y135Stop | 522T > A |
| | Y304C | 1028A > G |
| | Y304Stop | 1029T > G |
| | 241-242M ins | 842insTGA |
| | Splice | IVS10 + 2t > a |
| | Splice | IVS10 + 2t > g |
| | Splice | IVS2 + 1g > a |
| | Splice | IVS2 + 1g > t |
| | Splice | IVS4-2A > G-203a > g |
| | Splice | IVS5 + 1g > t |
| | Stop | 337A > T |
| Other GBA mutations | 36delT | 222-224del TAC |
| | A90T | 385G > A |
| | A232G | 812C > G |
| | C342G | 1141T > G |
| | C342R | 1141T > C |
| | D127V | 497A > T |
| | D140H | 535G > C |
| | D409G | 1343A > G |
| | F37V | 226T > G |
| | F216Y | 764T > A |
| | F397S | 1307T > C |
| | G46E | 254G > A |
| | G189V | 683G > T |
| | G377S | 1246G > A |
| | I402T | 1322T > C |
| | K79N | 354G > C |
| | K413Q | 1354A > C |
| | L66P | 314T > C |
| | L371V | 1228C > G |
| | M123V | 484A > G |
| | N117D | 466A > G |
| | N188S | 680A > G |
| | N370S | 1226A > G |
| | N396T | 1304A > C |
| | P122S | 481C > T |
| | P159L | 593C > T |
| | P159T | 592C > A |
| | P266R | 914C > G |
| | P289L | 983C > T |
| | P401L | 1319C > T |
| | P289L | 983C > T |
| | R48W | 259C > T |
| | R170C | 625C > T |
| | R170P | 626G > C |
| | R359Q | 1193G > A |
| | R433G | 1414A > G |
| | R496C | 1603C > T |
| | R496H | 1604G > A |
| | S271N | 929G > A |
| | S364T | 1208G > C |
| | S366G | 1213A > G |
| | T134I | 518C > T |
| | V15L | 160G > T |
| | V352L | 1171G > C |
| | V375L | 1240G > T |
| | W312C | 1053G > T |
| Sequence variants where | E326K | 1093G > A |
| history of RBD is required | T369M | 1223C > T |

^{a}Consult was required to determine eligibility for participants with GBA mutations not present in this list. cDNA = complementary deoxyribonucleic acid; GBA = glucocerebrosidase (glucosylceramidase beta) gene; RBD = rapid eye movement sleep behaviour disorder

Participants could continue concomitant symptomatic PD medications if the regimens were stable for ≥30 days (≥60 days for rasagiline) before randomisation.

### Exclusion Criteria

Key exclusion criteria included secondary or atypical parkinsonism (the manifestation of parkinsonism due to drugs or toxins), the presence of leucine rich repeat kinase 2 gene (LRRK2) G2019S mutation, carrying biallelic GBA mutations (i.e. GD diagnosis), patients with GD as defined by clinical signs and symptoms (i.e. hepatosplenomegaly, cytopenia, skeletal disease) and/or marked deficiency of GCase activity compatible with GD, a Montreal Cognitive Assessment [MoCA] score <20 at baseline (Nasreddine Z S et al., J Am Geriatr Soc 2005; 53: 695-9), past surgical history of deep brain stimulation, and the presence of any medical disorders and/or clinically relevant findings that could interfere with study-related procedures (e.g. conditions that preclude the safe performance of routine lumbar puncture (LP), such as prohibitive lumbar spinal disease, bleeding diathesis, or clinically significant coagulopathy or thrombocytopenia).

Further exclusion criteria included: current treatment with anticoagulants (e.g. coumadin, heparin) that might preclude safe completion of the LP; any medical disorders and/or clinically relevant findings in the physical examination, medical history, or laboratory assessments that, in the opinion of the investigator, could interfere with study-related procedures (e.g. heart failure, hypokalemia, etc.); presence of severe depression as measured by BDI-II>28 and/or a history of a major affective disorder within 1 year of screening examination; a history of drug and/or alcohol abuse within the past year prior to the first screening visit; baseline MRI without contrast showing a structural abnormality that is a possible aetiology of PD related signs and symptoms; current participation in another investigational interventional study; documented diagnosis of any of hepatitis B, hepatitis C, human immunodeficiency virus 1 or 2; use of any medication specifically used for treating memory dysfunction (e.g. cholinesterase inhibitors, or memantine), or use of strong or moderate inducers or inhibitors of CYP3A4, within 30 days or 5 half-lives prior to randomisation, whichever is longer; use of antipsychotics, modafinil, armodafinil, metoclopramide, alpha-methyldopa, methylphenidate, reserpine, or amphetamine derivatives within 6 months prior to DAT neuroimaging evaluation; use of an investigational medicinal product, including ambroxol, within 3 months or 5 half-lives, whichever is longer, before study inclusion; use of concomitant medications that prolong the time from ECG Q wave to the end of the T wave or corrected T wave corresponding to electrical systole (QT/QTc interval); a marked baseline prolongation of QT/QTc interval on screening ECG (such as a QTc interval >450 msec in male subjects and >470 msec in female subjects); liver enzymes (ALT/AST) or total bilirubin >2 times the ULN at the time of screening (participants with Gilbert's disease are excluded only from Part 1 participation); renal insufficiency as defined by creatinine >1.5 times ULN at the screening visit; use of concomitant symptomatic PD medication(s) (such as dopamine precursors (e.g. levodopa/carbidopa), dopamine agonists (e.g. ropinirole, bromocriptine, cabergoline, pergolide, pramipexole, and apomorphine), MAO B inhibitors (e.g. rasagiline and selegiline), anticholinergics (e.g. artane, cogentin), adamantane derivatives, and acetylcholinesterase inhibitors), where the treatment regimen with the symptomatic PD medication(s) is not stable for ≥30 days, or not stable for ≥60 days in the case of MAO B inhibitors (particularly rasagiline), before randomisation; presence, according to World Health Organization Grading, of cortical cataract >one quarter of the lens circumference (Grade cortical cataract-2) or a posterior subcapsular cataract >2 mm (Grade posterior subcapsular cataract-2); current receipt of potentially cataractogenic medications, including a chronic regimen (more frequently than every 2 weeks) of any dose or route of corticosteroids or any medication that may cause cataract or worsen the vision of participants with cataract (e.g. glaucoma medications) according to the prescribing information; and pregnancy, lactation, or breast-feeding.

### Clinical Trial

In part 1 of the trial, 29 participants were enrolled from 13 sites, including 4 Japanese sites, and 9 sites in 5 countries outside of Japan (Germany, Portugal, Spain, Sweden, and US). Since the risk of PD due to GBA mutations is higher in Japan than in other countries (Sidransky et al., N Engl J Med 2009; 361: 1651-61; Mitsui J et al., Arch Neurol 2009; 66: 571-6), Japanese participants (n=12) were evaluated separately as requested by the Japanese Pharmaceuticals and Medical Devices Agency, to compare pharmacokinetics and pharmacodynamics of Compound 2, and efficacy and safety outcomes, in this population to those observed in the rest of the world (ROW) population (17 participants).

Participants were randomised to 3 doses of once-daily orally administered Compound 2 or placebo, in sequential cohort design, and were followed up to 36 weeks (52 weeks for Japanese participants). Participants in the low-dose group received 4 mg of Compound 2 as a once daily oral dose; participants in the mid-dose group received 8 mg of Compound 2 a once daily oral dose; and participants in the high-dose group received 15 mg of Compound 2 a once daily oral dose.

The primary endpoint was safety and tolerability of Compound 2 compared to placebo. In addition, Compound 2 pharmacokinetics and pharmacodynamics were evaluated.

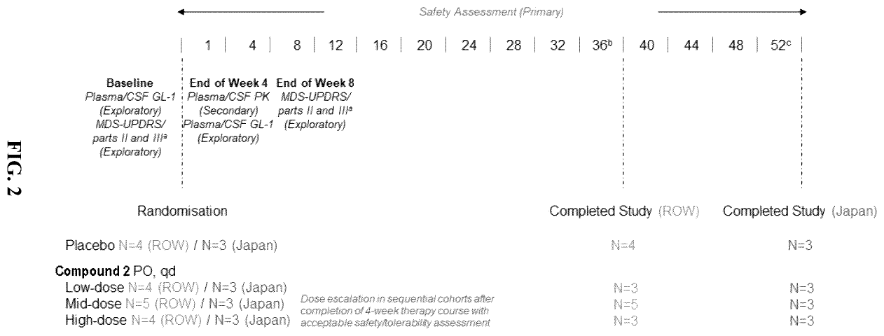

A total of 9 Japanese (low-dose: n=3; mid-dose: n=3; high-dose: n=3) and 13 ROW (low-dose: n=4; mid-dose: n=5; high-dose: n=4) participants recruited in part 1 were randomised to receive Compound 2, with 3 Japanese and 4 ROW participants randomised to placebo (FIG. 1 and FIG. 2).

The mean time since motor symptom onset and mean time since diagnosis were similar between the Japanese and ROW groups. Numerical differences were observed between groups in other baseline and disease characteristics (Table 2).

TABLE 2
Baseline characteristics in part 1 of the trial

| | Japan | | ROW | |
|---|---|---|---|---|
| | (N = 12) | | (N = 17) | |
| Age (y), mean (SD) | 54.3 | (8.6) | 58.4 | (7.9) |
| Sex, n (%) | | | | |
| Male | 7 | (58.3) | 13 | (76.5) |
| Race, n (%) | | | | |
| White | 0 | | 17 | (100) |
| Asian | 12 | (100) | 0 | |
| Time since symptoms onset (y) | | | | |
| Mean (SD) | 6.7 | (3.8) | 6.7 | (4.0) |
| Median (min, max) | 5.0 | (3, 14) | 6.0 | (2, 18) |
| Time since diagnosis (y) | | | | |
| Mean (SD) | 5.2 | (4.0) | 5.2 | (4.4) |
| Median (min, max) | 3.5 | (1, 14) | 4.7 | (0, 18) |
| Predominant symptoms at onset, n (%) | | | | |
| Rigidity/bradykinesia | 8 | (66.7) | 5 | (29.4) |
| Tremor | 4 | (33.3) | 12 | (70.6) |
| Family history of PD, n (%) | | | | |
| Yes | 3 | (25.0) | 7 | (41.2) |
| No | 9 | (75.0) | 10 | (58.8) |
| MoCA total score | | | | |
| Mean (SD) | 25.3 | (4.0) | 27.4 | (2·6) |
| MDS-UPDRS Part II + Part III score^{a} | | | | |
| Mean (SD) | 45.9 | (12.7) | 44.6 | (21.9) |
| Mutations | | | | |
| Any GBA mutation, n (%) | 12 | (100) | 17 | (100) |
| Any severe^{b} GBA mutation, n (%) | 9 | (75.0) | 7 | (41.2) |
| L444P (p.Leu483Pro) | 5 | (41.7) | 4 | (23.5) |
| 84GG (p.L29Afs*18) | 0 | | 3 | (17.6) |
| A456P (p.Ala495Pro) | 2 | (16.7) | 1 | (5.9) |
| R120W (p.Arg159Trp) | 3 | (25.0) | 0 | |
| D409H (p.Asp448His) | 1 | (8.3) | 0 | |
| Any other^{c} GBA mutation, n (%) | 3 | (25.0) | 10 | (58.8) |

| | | | |
|---|---|---|---|
| N370S (p.Asn409Ser) | 0 | 7 | (41.2) |
| E326K (p.Glu365Lys) | 0 | 3 | (17.6) |

| | | | |
|---|---|---|---|
| R496C (p.Arg535Cys) | 2 | (16.7) | 0 |
| G193W (p.Gly232Trp) | 1 | (8.3) | 0 |

DOPA = dihydroxyphenylalanine; GBA = glucocerebrosidase (glucosylceramidase beta) gene; max = maximum; MDS-UPDRS = Movement Disorder Society-Unified Parkinson′s Disease Rating Scale; min minimum; MoCA = Montreal Cognitive Assessment; PD = Parkinson′s disease; ROW = rest of the world; y = years.
^{a}Part II (motor experiences of daily living [13 items]) and Part III (motor examination [18 items]).
^{b}Severe GBA mutations are categorised as those that cause Gaucher Disease types 2 and 3.
^{c}Other, non-severe GBA mutations included mild GBA mutations that have been associated with GD type 1.

More Japanese participants presented with rigidity/bradykinesia versus ROW participants, and fewer had tremors. Fewer Japanese participants had a family history of PD versus ROW participants. Mean baseline MoCA scores were similar between the Japanese and ROW cohorts. Mean (standard deviation [SD]) MDS-UPDRS (parts II and III) scores at baseline were 45.9 (12.7) and 44.6 (21.9) for Japanese and ROW participants, respectively. Of the ROW participants, 7/17 (41.2%) had a severe GBA mutation, including L444P (4 participants), 84GG (3 participants), and A456P (1 participant), and 10/17 (58.8%) carried other GBA mutations, including N370S (7 participants), and the variant E326K (3 participants). The proportion of participants having a severe mutation was higher in the Japanese cohort (9/12 [75.0%], including L444P [5 participants], R120W [3 participants], A456P [2 participants], and D409H [1 participant]), whereas fewer Japanese participants had other mutations (3/12 [25.0%], including R496C [2 participants], and G193W [1 participant]). In all Compound 2-treated participants, median (interquartile range [IQR]) duration of exposure at low, mid, and high doses, was 35.7 (32.0; 40.1) weeks, 20.3 (17.5; 24.1) weeks, and 8.3 (8.0; 12.1) weeks, respectively.

The trial allowed for participants to continue or receive as-needed concurrent symptomatic PD medications, which were recorded at the time of the screening visit and during the study (Table 3). All Japanese participants and 15/17 (88.2%) of ROW participants received concomitant symptomatic medications summarised in Table 3.

TABLE 3
List of concomitant symptomatic PD medications

| | |
|---|---|
| Japan | ROW |
| (N = 12) | (N = 17) |

| | | | | |
|---|---|---|---|---|
| Any concomitant symptomatic | 12 | (100) | 15 | (88.2) |
| PD medication^{a}, n (%) | | | | |
| DOPA and DOPA derivatives | 12 | (100) | 12 | (70.6) |
| Dopamine agonists | 9 | (75.0) | 11 | (64.7) |
| Monoamine oxidase B inhibitors | 1 | (8.3) | 11 | (64.7) |
| Tertiary amines | 2 | (16.7) | 4 | (23.5) |
| Other dopaminergic agents | 1 | (8.3) | 3 | (17.6) |
| Adamantane derivatives | 5 | (41.7) | 1 | (5.9) |

DOPA = dihydroxyphenylalanine; PD = Parkinson′s disease; ROW = rest of the world.
^{a}Medication can be counted in more than 1 therapeutic class, and an individual participant can receive more than 1 concomitant symptomatic PD medication.

DOPA and DOPA derivatives included levodopa, carbidopa, benserazide, ledopsan, madopar, sinemet, and stalevo. Dopamine agonists included pramipexole, ropinirole, rotigotine, apomorphine, and cabergoline. Monoamine oxidase B inhibitors included selegiline, rasagiline, and safinamide. Tertiary amines included trihexyphenidyl, and bornaprine. Other dopaminergic agents included entcapone, and opicapone. Adamantane derivatives included amantadine.

Other prior medications used by study participants up to 3 months prior to first Compound 2 intake included prolactine inhibitors (e.g. cabergoline), drugs used in erectile dysfunction (e.g. apomorphine), and antidotes (e.g. apomorphine).

### Statistical Analysis

Analyses were based on all available data from participants who enrolled in part 1 of the trial and were randomised to receive either Compound 2 or placebo for up to 52 weeks for Japanese participants and 36 weeks for the ROW population. As part 1 was an exploratory, dose-escalation study, the sample size was not based on a statistical power calculation. Participants included in the primary safety analyses were those who were randomized and received at least one dose of Compound 2 or placebo. A data monitoring committee was responsible for overseeing the safety of patients and the risk/benefit ratio throughout the study. Duration of exposure to Compound 2 was different among the doses, as per the sequential cohort design, with the low- and high-dose cohorts commencing the study first and last, respectively. For pharmacokinetics, plasma parameters were calculated using noncompartmental methods, from plasma Compound 2 concentrations obtained after single dose administration. AUC₀₋₂₄ and AUC₀₋₄₈ were calculated using the trapezoidal method over a predefined time period of 24 hours or 48 hours, respectively. CLₛₛ/F was calculated using the following equation: CLₛₛ/F=Dose/AUC_{τ}, where τ is the dosing interval. For pharmacodynamics, percent reduction of plasma or CSF GL-1 from baseline was calculated as (value−baseline)/baseline×100. Formal statistical testing was not conducted on MDS-UPDRS scale assessments.

### Example 3: Compound 2 was Well Tolerated in Patients with PD

The primary outcome in part 1 of trial was an assessment of safety and tolerability of orally administered Compound 2 versus placebo through Week 4 (primary analysis period for each sequential cohort). In addition, safety assessments were recorded for every 4 weeks until completion. Safety was assessed by physical examination; neurological examination; clinical laboratory evaluations conducted at a central laboratory, including haematology, biochemistry, urinalysis, and serology tests; vital signs; assessment of AEs and concomitant medication; ophthalmological examination; and electrocardiogram. AEs, serious Aes, and AEs of special interest (AESI) were recorded throughout the study. Serious AEs were defined as fatal, or life threatening, requiring or prolonging inpatient hospitalisation, or disabling/incapacitating, congenital anomalies, or medically important events (e.g. malignancies or chronic neurodegenerative diseases). AESI included new or worsening lens opacities and cataracts (as assessed by ophthalmology evaluations at screening and at Weeks 4, 12, 28, 36 [Weeks 4, 12, 36, 52 for Japanese participants]), pregnancy events, increase of alanine aminotransferase, or symptomatic overdose with study drug.

During the study, 29 participants were randomised to Compound 2 (Japan, n=9; ROW, n=13) or placebo (n=3; n=4). Eight (89%) Japanese and 12 (92%) ROW Compound 2-treated participants experienced ≥1 adverse event (AE) versus 2 (67%) and 4 (100%) participants from the respective placebo groups. Most AEs were mild or moderate, and no serious AEs or deaths occurred. Two Compound 2-treated ROW participants discontinued due to AEs (confusional state and panic attack). Table 4 shows AE data collected during the treatment-emergent period, defined as the period from first intake of treatment to last intake of treatment+6 weeks.

TABLE 4
AEs throughout part 1 of the trial^{a}

| Number (%) of | Japan (N = 12) | | | | ROW (N = 17) | | | |
|---|---|---|---|---|---|---|---|---|
| participants with AEs | | Compound 2 | | | | Compound 2 | | |
| by MedDRA Primary | Placebo | Low | Mid | High | Placebo | Low | Mid | High |
| System Organ Class | (n = 3) | (n = 3) | (n = 3) | (n = 3) | (n = 4) | (n = 4) | (n = 5) | (n = 4) |
| Any AE | 2 | 3 | 3 | 2 | 4 | 4 | 4 | 4 |
| | (66.7) | (100) | (100) | (66.7) | (100) | (100) | (80.0) | (100) |
| Ear and labyrinth | 0 | 1 | 0 | 0 | 0 | 0 | 0 | 1 |
| disorders | | (33.3) | | | | | | (25.0) |
| Eye disorders | 0 | 1 | 1 | 0 | 0 | 2 | 1 | 0 |
| | | (33.3) | (33.3) | | | (50.0) | (20.0) | |
| Gastrointestinal | 0 | 3 | 0 | 1 | 2 | 2 | 2 | 0 |
| disorders | | (100) | | (33.3) | (50.0) | (50.0) | (40.0) | |
| General disorders and | 0 | 0 | 0 | 0 | 0 | 0 | 1 | 1 |
| administration site | | | | | | | (20.0) | (25.0) |
| conditions | | | | | | | | |
| Infections and | 1 | 2 | 0 | 1 | 0 | 0 | 0 | 1 |
| infestations | (33.3) | (66.7) | | (33.3) | | | | (25.0) |
| Injury, poisoning, and | 0 | 0 | 1 | 2 | 0 | 1 | 1 | 1 |
| procedural | | | (33.3) | (66.7) | | (25.0) | (20.0) | (25.0) |
| complications | | | | | | | | |
| Investigations | 1 | 0 | 0 | 0 | 0 | 0 | 1 | 0 |
| | (33.3) | | | | | | (20.0) | |
| Musculoskeletal and | 0 | 0 | 0 | 0 | 0 | 2 | 1 | 0 |
| connective tissue | | | | | | (50.0) | (20.0) | |
| disorders | | | | | | | | |
| Nervous system | 0 | 1 | 0 | 0 | 3 | 1 | 1 | 0 |
| disorders | | (33.3) | | | (75.0) | (25.0) | (20.0) | |
| Psychiatric disorders | 0 | 1 | 2 | 0 | 0 | 3 | 3 | 2 |
| | | (33.3) | (66.7) | | | (75.0) | (60.0) | (50.0) |
| Renal and urinary | 1 | 0 | 0 | 1 | 0 | 0 | 0 | 1 |
| disorders | (33.3) | | | (33.3) | | | | (25.0) |
| Respiratory, thoracic, | 0 | 0 | 0 | 0 | 0 | 0 | 0 | 1 |
| and mediastinal | | | | | | | | (25.0) |
| disorders | | | | | | | | |
| Skin and | 0 | 0 | 1 | 0 | 0 | 0 | 0 | 1 |
| subcutaneous tissue | | | (33.3) | | | | | (25.0) |
| disorders | | | | | | | | |
| Vascular disorders | 0 | 0 | 0 | 0 | 0 | 0 | 0 | 1 |
| | | | | | | | | (25.0) |
| AEs leading to study | 0 | 0 | 0 | 0 | 0 | 1 | 0 | 1 |
| discontinuation | | | | | | (25.0) | | (25.0) |
| Confusional state | 0 | 0 | 0 | 0 | 0 | 1 | 0 | 0 |
| | | | | | | (25.0) | | |
| Panic attack | 0 | 0 | 0 | 0 | 0 | 0 | 0 | 1 |
| | | | | | | | | (25.0) |

^{a}Safety outcomes were assessed at Day 1, Day 2, Day 3, Week 2, Week 4, then every 4 weeks up to 36 weeks (up to 52 weeks for Japanese participants). AEs included in this table are those which occurred during the treatment-emergent period (defined as the period from first intake of treatment to last intake of treatment + 6 weeks). AE = adverse event; MedDRA = Medical Dictionary for Regulatory Activities.

Nervous system disorders and gastrointestinal events were frequent in both placebo and Compound 2-treated participants, consistent with common PD symptoms (DeMaagd et al., P T2015; 40: 504-32; Poirier et al., Parkinsons Dis 2016; 2016: 6762528). A high frequency of psychiatric disorders was observed in Compound 2-treated patients (Table 5). These disorders included known psychiatric clinical manifestations with high prevalence rate in PD patients, such as insomnia, anxiety, depression, and hallucinations (Grover S et al., J Neurosci Rural Pract 2015; 6: 65-76). The relatively high frequency of neurologic, psychiatric, and gastrointestinal disorders in part 1 participants might be attributed in part to concurrent PD medication use, including dopamine agonists (Ceravolo R et al., Expert Opin Drug Saf 2016; 15:181-98).

TABLE 5
Psychiatric disorders in participants^{a}
Japan (N = 12)

| | | Compound 2 | | |
|---|---|---|---|---|
| | Placebo | Low | Mid | High |
| | (n = 3) | (n = 3) | (n = 3) | (n = 3) |
| Psychiatric Disorders, | 0 | 1 (33.3) | 2 (66.7) | 0 |
| number (%)^{b} | | | | |
| Delirium | 0 | 0 | 1 (33.3) | 0 |
| Hallucination | 0 | 0 | 1 (33.3) | 0 |
| Insomnia | 0 | 1 (33.3) | 0 | 0 |

ROW (N = 17)

| | | Compound 2 | | |
|---|---|---|---|---|
| | Placebo | Low | Mid | High |
| | (n = 4) | (n = 4) | (n = 5) | (n = 4) |
| Psychiatric Disorders, | 0 | 3 (75.0) | 3 (60.0) | 2 (50.0) |
| number (%)^{b} | | | | |
| Anxiety | 0 | 0 | 0 | 2 (50.0) |
| Depressed mood | 0 | 1 (25.0) | 1 (20.0) | 0 |
| Hallucination (visual) | 0 | 0 | 2 (40.0) | 0 |
| Confusional state | 0 | 1 (25.0) | 0 | 0 |
| Depression | 0 | 1 (25.0) | 0 | 0 |
| Panic attack | 0 | 0 | 0 | 1 (25.0) |
| Sleep disorder | 0 | 0 | 1 (20.0) | 0 |

^{a}Safety outcomes were assessed every 4 weeks for a maximum of 36 weeks (up to 52 weeks for Japanese participants), until the completion of Part 1;
^{b}By MedDRA Primary System Organ Class. MedDRA = Medical Dictionary for Regulatory Activities.

These findings demonstrate that Compound 2 was well tolerated in patients with PD.

### Example 4: Pharmacokinetics of Compound 2 in Plasma and CSF

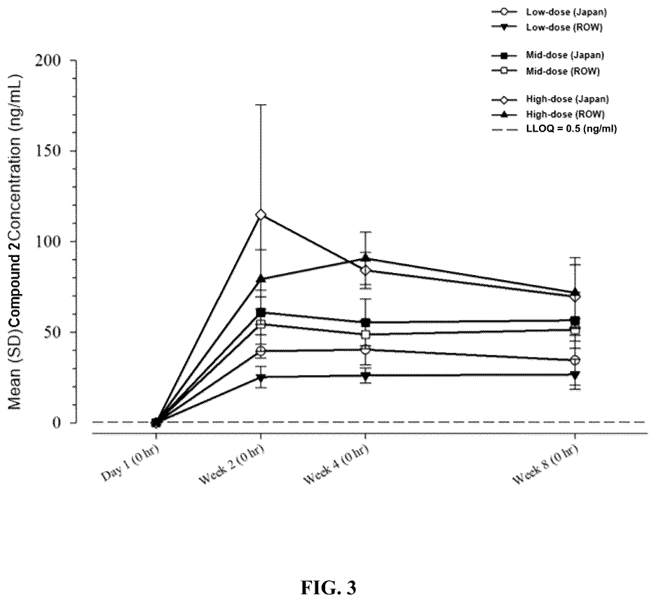

The secondary outcome was to evaluate the pharmacokinetic profile of Compound 2 in plasma and cerebrospinal fluid (CSF) over a 4-week period. Plasma pharmacokinetic parameters included maximum plasma concentration (Cₘₐₓ), time to Cₘₐₓ (tₘₐₓ), and area under the plasma concentration versus time curve from 0 to 24 h (AUC₀₋₂₄). All parameters were assessed at Week 0 and Week 4 after treatment. In addition, AUC₀₋₄₈ was assessed at Week 0, trough plasma concentration observed just before treatment administration during repeated dosing (C_{trough}) was assessed at Weeks 2, 4, and 8 (FIG. 3), and total systemic clearance from plasma at steady state (CL_{SS}/F) was assessed at Week 4. Plasma samples were collected at 1 hour pre-dose and 1, 2, 4, 8, 24, and 48 hours postdose. Compound 2 concentration in plasma was determined using a liquid chromatography tandem mass spectrometry method with a lower limit of quantification of 0.500 ng/mL. CSF samples were collected by lumbar puncture during the screening period (within 14 days prior to randomisation) or on the day of randomisation, and at Week 4, within 2 to 4 hours postdose. Compound 2 concentration in CSF was determined using a liquid chromatography tandem mass spectrometry method with a lower limit of quantification of 0.100 ng/mL.

Pharmacokinetic data at Day 1 and Week 4 after Compound 2 treatment are reported in Tables 6a and 6b.

TABLE 6a
Plasma pharmacokinetic parameters of Compound 2 at Day 1

| | Japan (N = 9) | | | ROW (N = 13) | | |
|---|---|---|---|---|---|---|
| | Compound 2 | | | Compound 2 | | |
| | Low | Mid | High | Low | Mid | High |
| | (n = 3) | (n = 3) | (n = 3) | (n = 4) | (n = 5) | (n = 4) |
| Cₘₐₓ (ng/mL), | 21.8 ± | 31.3 ± | 54.1 ± | 13.8 ± | 26.3 ± | 47.4 ± |
| mean ± SD | 2.44 | 10.5 | 11.7 | 2.27 | 12.3 | 6.03 |
| Geometric | 21.7 | 29.9 | 53.3 | 13.6 | 23.8 | 47.1 |
| mean | (11.2) | (33.6) | (21.6) | (16.5) | (46.7) | (12.7) |
| (CV %) | | | | | | |
| tₘₐₓ (h), | 4.33 | 4.02 | 4.10 | 4.07 | 4.17 | 3.94 |
| median | (4.00- | (2.08- | (4.00- | (1.00- | (2.00- | (2.25- |
| (range) | 7.17) | 7.00) | 7.17) | 8.00) | 8.00) | 7.00) |
| AUC₀₋₂₄ | | | | | | |
| (ng · h/mL), | 393 ± | 623 ± | 990 ± | 255 ± | 457 ± | 873 ± |
| mean ± SD | 52.8 | 211 | 203 | 59.6 | 186 | 108 |
| Geometric | 390 | 596 | 977 | 249 | 422 | 868 |
| mean | (13.4) | (33.9) | (20.5) | (23.4) | (40.8) | (12.4) |
| (CV %) | | | | | | |
| AUC₀₋₄₈ | 661 ± | 1130 ± | 1590 ± | 431 ± | 776 ± | 1450 ± |
| (ng · h/mL), | 47.4 | 431 | 312 | 111 | 269 | 127 |
| mean ± SD | | | | | | |
| Geometric | 660 | 1070 | 1570 | 419 | 735 | 1440 |
| mean | (7.2) | (38.3) | (19.6) | (25.7) | (34.6) | (8.8) |
| (CV %) | | | | | | |

AUC₀₋₂₄ = area under the plasma concentration versus time curve from 0 to 24 h; AUC₀₋₄₈ = area under the plasma concentration versus time curve from 0 to 48 h; Cₘₐₓ = maximum observed concentration; CV % = coefficient of variation; h = hours; tₘₐₓ = time to maximum concentration.
^{a}Plasma samples were collected at pre-dose and 1, 2, 4, 8, 24, 48 hours postdose.

TABLE 6b
Plasma and CSF pharmacokinetic concentrations
of Compound 2 at Week 4

| Japan (N = 9) | | | ROW (N = 13) | | |
|---|---|---|---|---|---|
| Compound 2 | | | Compound 2 | | |
| Low | Mid | High | Low | Mid | High |
| (n = 3) | (n = 3) | (n = 3) | (n = 4) | (n = 5) | (n = 4) |

Plasma pharmacokinetic parameters at week 4^{a}

| | | | | | | |
|---|---|---|---|---|---|---|
| Cₘₐₓ (ng/ml), | 56.3 ± | 83.7 ± | 145 ± | 41.6 ± | 77.8 ± | 136 ± |
| mean ± SD | 3.64 | 15.1 | 17.6 | 7.39 | 20.1 | 13.4 |
| Geometric | 56.2 | 82.8 | 144 | 41.0 | 75.8 | 135 |
| mean | (6.5) | (18.0) | (12.2) | (17.8) | (25.8) | (9.9) |
| (CV %) | | | | | | |
| Fold increase | — | 1.47 | 2.56 | — | 1.85 | 3.29 |
| vs low-dose | | | | | | |
| tₘₐₓ (h), | 4.33 | 2.12 | 2.10 | 1.96 | 3.55 | 2.89 |
| median | (4.33- | (1.92- | (1.93- | (1.00- | (2.00- | (2.00- |
| (range) | 8.00) | 3.58) | 3.70) | 24.00) | 8.03) | 3.98) |
| AUC₀₋₂₄ | 1130 ± | 1650 ± | 3150 ± | 766 ± | 1510 ± | 2510 ± |
| (ng · h/mL), | NA^{b} | 382 | NA^{c} | 84.0 | 305 | 369 |
| mean ± SD | | | | | | |
| Geometric | 1130 | 1620 | 3140 | 763 | 1490 | 2490 |
| mean | (NA) | (23.1) | (NA) | (11.0) | (20.2) | (14.7) |
| (CV %) | | | | | | |
| Fold increase | — | 1.43 | 2.78 | — | 1.95 | 3.26 |
| vs low dose | | | | | | |
| CLₛₛ/F (mL/h), | 3540 ± | 5010 ± | 4780 ± | 5270 ± | 5450 ± | 6100 ± |
| mean ± SD | NA^{b} | 1070 | NA^{c} | 626 | 1010 | 1050 |
| Geometric | 3540 | 4930 | 4770 | 5240 | 5370 | 6030 |
| mean | (NA) | (21.3) | (NA) | (11.9) | (18.5) | (17.2) |
| (CV %) | | | | | | |
| Fold increase | — | 1.39 | 1.35 | — | 1.02 | 1.15 |
| vs low dose | | | | | | |

CSF pharmacokinetic concentrations at week 4 (2-4 h postdose)

| | | | | | | |
|---|---|---|---|---|---|---|
| CSF | 2.11 | 4.33 | 10.1 | 1.85 | 3.15 | 5.96 |
| concentration | (7.8) | (37.3) | (9.1) | (6.2)^{d} | (32.1)^{d} | (15.2)^{e} |
| (ng/mL), | | | | | | |
| mean (CV %) | | | | | | |
| Fold increase | — | 2.05 | 4.79 | — | 1.70 | 3.22 |
| vs low dose | | | | | | |

AUC₀₋₂₄ = area under the plasma concentration versus time curve from 0 to 24 h; Cₘₐₓ = maximum observed concentration; CL_{SS}/F = total systemic clearance at steady state; CSF = cerebrospinal fluid; CV % = geometric coefficient of variation; h = hours; NA = not applicable; ROW = rest of the world; tₘₐₓ = time to maximum concentration.
^{a}Plasma samples were collected at 1 h pre-dose and 1, 2, 4, 8, and 24 h postdose;
^{b}n = 1;
^{c}n = 2;
^{d}3 participants (1 from the low-dose group and 2 from the mid-dose group) were not included in the CSF pharmacokinetic analysis because Week 4 CSF sample was collected before dose instead of at the protocol-specified postdose time;
^{e}Week 4 CSF sample was not collected for 1 participant.

Compound 2 concentration in plasma and CSF increased in a close to dose-proportional manner in both the Japanese and ROW cohorts (Table 6b). At Week 4, Compound 2 was absorbed in plasma with median tₘₐₓ values of 2.10-4.33 hours in Japanese participants, and 1.96-3.55 hours in ROW participants, across all doses tested. The Compound 2 dosage increase between low- and high-dose treated participants resulted in a 2.56-fold increase in geometric mean Cₘₐₓ in plasma in the Japanese participant cohort, and a 3.29-fold increase in ROW participants. Increases were also observed in the plasma geometric mean AUC₀₋₂₄, and CLₛₛ/F. Mean CSF concentration of Compound 2 at Week 4 increased 4.79-fold in Japanese participants and 3.22-fold in ROW participants between low- and high-dose treated participants. At Weeks 2, 4, and 8, C_{trough} was increased in a dose-proportional manner (FIG. 3). Steady state exposure pharmacokinetic parameters were similar between the high-dose Compound 2-treated ROW participants (following 4 weeks of once daily dosing, mean (SD) Cₘₐₓ was 136 (13.4) ng/mL and AUC₀₋₂₄ was 2510 (369) ng·h/mL) and healthy individuals (142 (40.0) ng/mL and AUC₀₋₂₄ was 2420 (705.0) ng·h/mL).

### Example 5: Plasma and CSF GL-1 Levels Decreased from Baseline in Patients Treated with Compound 2 in a Dose-Dependent Manner

In exploratory analyses, Compound 2 pharmacodynamics in plasma and CSF were assessed by mean percent change in GL-1 levels from baseline through Week 4. GL-1 levels were assessed in plasma and CSF samples collected at the day of randomisation and Week 4, with plasma GL-1 levels also assessed at Week 2. Levels of glucosylsphingosine (GlcSph, lyso-GL1), a metabolic product of GL-1, were also measured in plasma and CSF, but most values were found to be below the limit of quantification of the assay, reflecting the fact that enrolled participants are heterozygous carriers of GBA mutations, and they do not have a GD diagnosis.

Concentrations of GL-1 in plasma and CSF were determined using a liquid chromatography tandem mass spectrometry method. Secondary pharmacokinetic outcomes were evaluated over a 4-week period of once-daily Compound 2 dosing (Table 7).

TABLE 7
Plasma and CSF glucosylceramide levels from baseline to Week 4

| | Japan (N = 12) | | | | ROW (N = 17) | | | |
|---|---|---|---|---|---|---|---|---|
| Measured GL-1 | | Compound 2 | | | | Compound 2 | | |
| levels | Placebo | Low | Mid | High | Placebo | Low | Mid | High |
| (mean(SD)) | (n = 3) | (n = 3) | (n = 3) | (n = 3) | (n = 4) | (n = 4) | (n = 5) | (n = 4)^{a} |
| Plasma GL-1 (μg/mL) | | | | | | | | |
| Baseline | 4.780 | 6.027 | 4.640 | 5.517 | 6.093 | 5.155 | 5.474 | 5.588 |
| | (0.989) | (1.066) | (0.685) | (1.586) | (1.081) | (0.952) | (0.569) | (1.121) |
| Week 2 | 5.423 | 2.230 | 1.507 | 1.231 | 5.558 | 2.340 | 1.644 | 1.469 |
| | (0.880) | (0.773) | (0.262) | (0.231) | (1.133) | (0.373) | (0.336) | (0.469) |
| Change from baseline | +0.643 | −3.797 | −3.133 | −4.285 | −0.535 | −2.815 | −3.830 | −4.119 |
| | (0.450) | (0.475) | (0.510) | (1.379) | (1.586) | (0.664) | (0.523) | (0.849) |
| Change from baseline (%) | +14.4 | −63.6 | −67.5 | −77.2 | −7.0 | −54.3 | −69.9 | −73.9 |
| | (11.7) | (7.1) | (3.9) | (2.8) | (23.1) | (4.8) | (5.9) | (5.9) |
| Week 4 | 4.863 | 2.097 | 1.340 | 1.149 | 6.220 | 2.093 | 1.578 | 1.253 |
| | (0.283) | (0.931) | (0.030) | (0.384) | (0.544) | (0.385) | (0.154) | (0.322) |
| Change from baseline | +0.083 | −3.930 | −3.300 | −4.368 | +0.128 | −3.063 | −3.896 | −4.335 |
| | (0.706) | (0.546) | (0.687) | (1.207) | (0.988) | (0.793) | (0.546) | (0.861) |
| Change from baseline (%) | +3.7 | −66.1 | −70.7 | −79.3 | +3.8 | −59.0 | −71.0 | −77.7 |
| | (13.9) | (10.2) | (4.1) | (1.2) | (14.8) | (6.4) | (3.9) | (2.7) |
| CSF GL-1 (ng/mL) | | | | | | | | |
| Baseline | 9.750 | 5.577 | 8.050 | 9.433 | 5.860 | 6.748 | 10.782 | 6.873 |
| | (4.303) | (2.215) | (3.557) | (3.892) | (1.703) | (2.912) | (10.041) | (5.659) |
| Week 4 | 9.253 | 3.843 | 3.027 | 2.770 | 6.528 | 4.315 | 4.406 | 1.623 |
| | (2.841) | (0.541) | (2.638) | (1.542) | (1.816) | (2.505) | (3.578) | (1.080) |
| Change from baseline | −0.497 | −1.733 | −5.023 | −6.663 | +0.668 | −2.433 | −6.376 | −5.250 |
| | (1.914) | (2.307) | (0.946) | (2.593) | (0.634) | (0.548) | (6.572) | (4.581) |
| Change from baseline (%) | −1.0 | −21.7 | −67.0 | −72.0 | +12.8 | −41.0 | −58.0 | −74.3 |
| | (15.6) | (39.7) | (15.6) | (9.9) | (13.6) | (18.1) | (11.5) | (4.3) |

^{a}CSF sample was not collected for 1 participant.

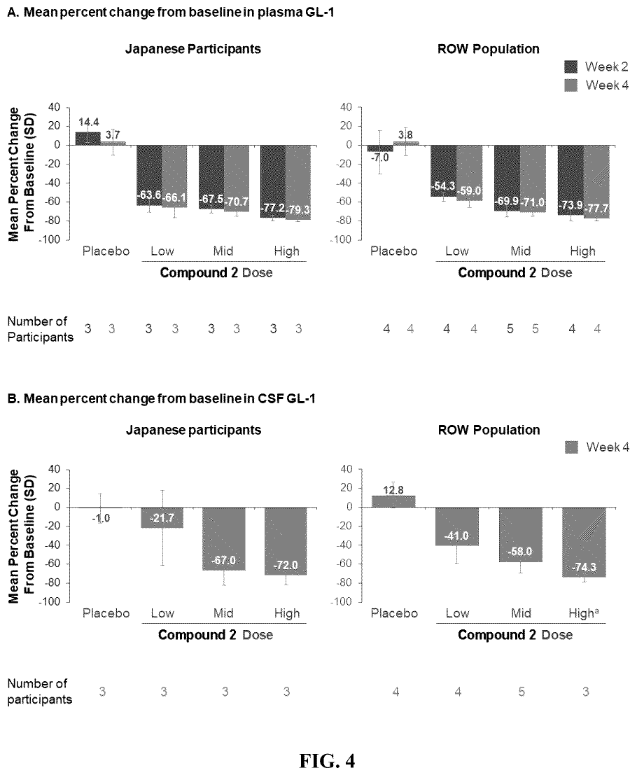

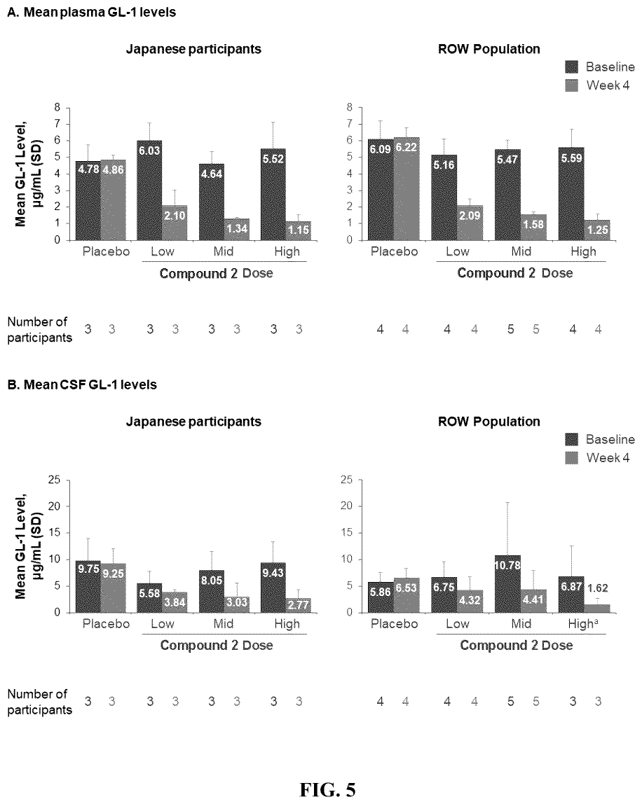

Plasma and CSF GL-1 levels decreased from baseline in Compound 2-treated patients in a dose-dependent manner over 4 weeks (FIG. 4; FIG. 5). For low-, medium-, and high-dose Compound 2, the respective mean reductions in plasma GL-1 at Week 4 were 66.1%, 70.7%, and 79.3% for the Japanese cohort, and 59.0%, 71.0%, and 77.7% for ROW participants; similar decreases in plasma GL-1 levels were observed at Week 2 in both cohorts. For low-, medium-, and high-dose Compound 2, the respective mean reductions in CSF GL-1 at Week 4 were 21.7%, 67.0%, and 72.0% for the Japanese cohort, and 41.0%, 58.0%, and 74.3% for ROW participants.

Exploratory analyses of the pharmacodynamic response of Compound 2 demonstrated a dose-dependent decrease of GL-1 levels in plasma and CSF samples over the same period. At the highest dose, a decrease of 72.0%-74.3% in CSF GL-1 levels from baseline was observed, indicating that Compound 2 is brain-penetrant on oral administration and can modify the glycosphingolipid profile in the brain and CSF. Sustained reduction of GL-1 levels in the CSF of participants in this study reflect sufficient target engagement of Compound 2, showing for the first time modulation of lipid profile in the central nervous system of PD patients.

An additional exploratory outcome was to assess the effect of Compound 2 on selected scales and questionnaires.

### Example 6: Effect of Compound 2 on MDS-UPDRS (Parts II and III)

The MDS-UPDRS is a revision of the Unified Parkinson's Disease Rating Scale (UPDRS) originally developed in the 1980s. It addresses several problematic areas of the original scale identified by an MDS review task force. The MDS-UPDRS was developed to evaluate various aspects of Parkinson's disease including non-motor and motor experiences of daily living and motor complications (Goetz et al., Mov Disord 2007; 22: 41-7, and Goetz et al., Mov Disord 2008; 23:2129-70). It includes a motor evaluation and characterizes the extent and burden of disease across various populations. The scale can be used in a clinical setting as well as in research (https://www.movementdisorders.org/MDS/MDS-Rating-Scales/MDS-Unified-Parkinsons-Disease-Rating-Scale-MDS-UPDRS.htm).

MDS-UPDRS (parts II [motor experiences of daily living; 13 items] and III [motor examination; 18 items]) scores are reported through Week 8. The MDS-UPDRS scale was assessed during the OFF state, with no PD medication taken for ≥12 hours prior.

The changes from baseline in MDS-UPDRS (parts II and III) scores at week 8 were assessed for participants in part 1 of the clinical study described in Example 2. The results are shown in Table 8 below.

TABLE 8
Changes from baseline in MDS-UPDRS (parts II and III) scores at week 8

| Placebo (N = 7) | | | Compound 2 (N = 22) | | |
|---|---|---|---|---|---|
| Japan | ROW | Combined | Japan | ROW | Combined |
| (n = 3) | (n = 4) | Japan + ROW | (n = 9) | (n = 13) | Japan + ROW |

MDS-UPDRS (part II + III) score

| | | | | | | |
|---|---|---|---|---|---|---|
| Change from | +0.3 | −3.0 | −1.6 | +0.7 | −4.5 | −2.9 |
| baseline at week | (2.5) | (9.6) | (7.1) | (10.7) | (13.3) | (12.2) |
| 8 (mean (SD)) | | | | | | |

Through week 8, mean (SD) changes from baseline in MDS-UPDRS (parts II and III) scores performed in the OFF state in Compound 2-treated Japanese and ROW participants were −0.7 (10.7), and −4.5 (13.3), respectively, compared to +0.3 (2.5), and −3.0 (9.6) points observed in the respective placebo groups.

1. A method of treating or preventing a neurodegenerative disease in a human subject by reducing glycosphingolipid concentration in brain tissue of the subject, whereby the concentration of glucosylceramide (GL-1) in the cerebrospinal fluid (CSF) of the subject is reduced by at least 30%, wherein the neurodegenerative disease is selected from Parkinson's disease (PD) and dementia with Lewy Bodies (DLB) and:
(a) wherein the subject does not have (e.g. has not been diagnosed as having, or being at risk of having) a lysosomal storage disease; and/or
(b) wherein the subject is a heterozygous carrier of one or more glucocerebrosidase 1 gene (GBA1) mutations;
the method comprising administering to the subject an effective amount of a compound of formula (I), 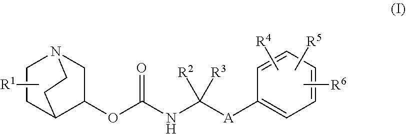
or a pharmaceutically acceptable salt or prodrug thereof, wherein:
R¹ is selected from hydrogen, halogen (e.g. fluorine), cyano, nitro, hydroxy, thio, amino, C₁₋₆-alkyl (e.g. methyl or ethyl), C₂₋₆-alkenyl, C₂₋₆-alkynyl, C₁₋₆-alkyloxy, C₂₋₆-alkenyloxy, and C₂₋₆-alkynyloxy, wherein said alkyl, alkenyl, alkynyl, alkyloxy, alkenyloxy, or alkynyloxy is optionally substituted with one or more (e.g. 1, 2, or 3) groups selected from halogen, cyano, nitro, hydroxy, thio, or amino;
R² and R³ are independently selected from C₁₋₃-alkyl, optionally substituted by one or more (e.g. 1, 2, or 3) halogens, or R² and R³ together form a cyclopropyl or cyclobutyl group, optionally substituted by one or more (e.g. 1 or 2) halogens;
R⁴, R⁵, and R⁶ are each independently selected from hydrogen, halogen, nitro, hydroxy, thio, amino, C₁₋₆-alkyl, and C₁₋₆-alkyloxy, wherein said alkyl or alkyloxy is optionally substituted by one or more (e.g. 1, 2, or 3) groups selected from halogen, hydroxy, cyano, and C₁₋₆-alkyloxy; and
A is a 5- or 6-membered aryl or heteroaryl group (e.g. phenyl or thiazolyl), optionally substituted with 1, 2, or 3 groups independently selected from halogen, hydroxy, thio, amino, nitro, C₁₋₆-alkyloxy, and C₁₋₆-alkyl.

2. The method of claim 1, wherein:
R¹ is selected from hydrogen, fluorine, methyl, and ethyl, wherein said methyl or ethyl is optionally substituted by 1 or 2 groups selected from halogen, hydroxy, thio, or amino;
R² and R³ are each independently selected from methyl and ethyl groups, optionally substituted with one or more fluorines;
R⁴ is selected from a halogen (e.g. fluorine), C₁₋₃-alkyl (e.g. methyl), and C₁₋₃-alkyloxy (e.g. methoxy or ethoxy), wherein said alkyl or alkyloxy is optionally substituted by one or more (e.g. 1, 2, or 3) groups selected from a halogen and C₁₋₃-alkyloxy (e.g. methoxy or ethoxy);
R⁵ and R⁶ are each hydrogen;
R⁴ is fluorine or 2-methoxyethoxy, and R⁵ and R⁶ are hydrogen;
R⁴ is positioned at the 4-position of the phenyl ring to which it is attached (i.e. para to the A substituent); and/or
A is phenyl, optionally substituted with 1, 2, or 3 groups independently selected from halogen, hydroxy, thio, amino, nitro, C₁₋₆alkyloxy, and C₁₋₆alkyl (e.g. methyl) or
A is a 5-membered heteroaryl group which contains 1 or 2 heteroatoms selected from N and S.

3-11. (canceled)

12. The method of claim 1, wherein said compound is a compound of formula (II), (III), (IV), (V), (VI), (VII), (VIII), (IX) or (XI), 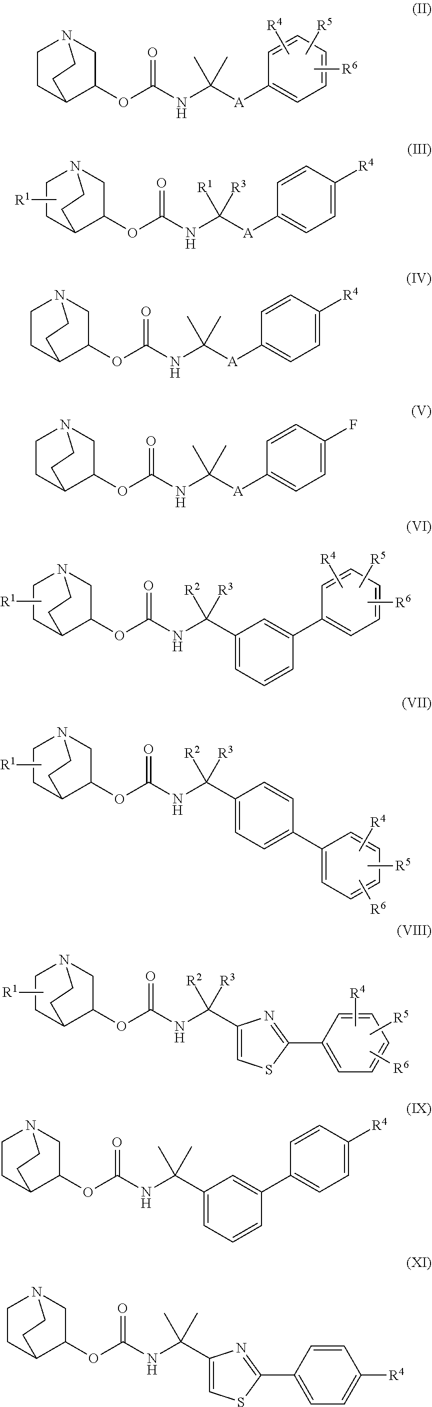
or a pharmaceutically acceptable salt or prodrug thereof.

13-16. (canceled)

17. The method of claim 1, wherein said compound is selected from: quinuclidin-3-yl (2-(4′-fluoro-[1,1′-biphenyl]-3-yl)propan-2-yl)carbamate; (S)-quinuclidin-3-yl (2-(2-(4-fluorophenyl)thiazol-4-yl)propan-2-yl)carbamate; (S)-quinuclidin-3-yl (2-(4′-(2-methoxyethoxy)-[1,1′-biphenyl]-4-yl)propan-2-yl)carbamate; and the pharmaceutically acceptable salts and prodrugs thereof.

18. (canceled)

19. (canceled)

20. The method of claim 1, wherein the compound is an acid addition salt form of (S)-quinuclidin-3-yl (2-(2-(4-fluorophenyl)thiazol-4-yl)propan-2-yl)carbamate, selected from the hydrochloride, hydroxysuccinate, and malate.

21. The method of claim 1, wherein the subject is a heterozygous carrier of one or more glucocerebrosidase 1 gene (GBA1) mutations.

22. The method of claim 21, wherein the one or more GBA1 mutations are selected from L444P, 84GG, A456P, R120W, D409H, E235A, E340A, N370S, E326K, R496C, G193W, T369M, R496H, and S271G.

23. The method of claim 1, wherein the brain tissue is (or comprises) neurons; and/or wherein said brain tissue is a neuron of the substantia nigra, cerebral cortex, hippocampus, frontal lobes, and/or temporal lobes of said subject.

24. (canceled)

25. (canceled)

26. The method of claim 1, wherein the method results in a reduction in GL-1 concentration in CSF of at least 40% (e.g. at least 50%, at least 60%, or at least 70%) and/or wherein the method results in the reduction in GL-1 concentration in the CSF within 3 months of commencing treatment (e.g. within 2 months, 4 weeks, 2 weeks, or 1 week of commencing treatment).

27. (canceled)

28. The method of claim 1, wherein: i) said subject has been diagnosed as having Parkinson's disease (e.g. early-stage Parkinson's disease) and wherein the method prevents, reduces, or reverses motor dysfunction (e.g. tremor), bradykinesia, rigidity, postural instability, and/or impaired balance; ii) said subject has been diagnosed as being at risk of developing Parkinson's disease, and wherein the method prevents or delays the onset and/or development of Parkinson's disease in the subject; or iii) said subject has a diagnosis of dementia with Lewy Bodies (DLB) and the method prevents, reduces, or reverses the progression of dementia in the subject.

29. (canceled)

30. (canceled)

31. The method of claim 28, wherein the subject has a diagnosis of Parkinson's disease and wherein the subject has at least one of the following characteristics: i) a family history of Parkinson's disease; ii) a baseline Montreal Cognitive Assessment (MoCA) score of ≤26 (for example, from 20 to 25); and iii) a Movement Disorder Society-Unified Parkinson's Disease Rating Scale (MDS-UPDRS) (Part II+III) score of at least 35; or wherein the subject has a diagnosis of early-stage Parkinson's disease characterized by: i) at least two of the following conditions: resting tremor, postural instability, akinesia/hypokinesia, and muscle rigidity; ii) a Hoehn and Yahr Scale stage of ≤2 at baseline; and/or iii) a Parkinson's diagnosis of ≥2 years.

32. (canceled)

33. The method of claim 28, wherein the subject has been treated historically with medications selected from dihydroxyphenylalanine (DOPA) or derivatives thereof (e.g. levodopa/carbidopa), monamine oxidase B inhibitors (e.g. rasagiline or selegiline), dopamine agonists (e.g. ropinirole, bromocriptine, cabergoline, pergolide, pramipexole, or apomorphine), Catechol-O-methyltransferase inhibitors (e.g. entacapone, tolcapone), anticholinergics (e.g. artane, cogentin), adamantane derivatives, and/or acetylcholinesterase inhibitors (e.g. tacrine, rivastigmine, galantamine, donepezil, or memantine).

34. (canceled)

35. (canceled)

36. A therapeutic method of reducing glycosphingolipid concentration in brain tissue of a human subject in need thereof, whereby the concentration of glucosylceramide (GL-1) in the cerebrospinal fluid (CSF) of the subject is reduced as a result of the method, wherein the subject does not have (e.g. has not been diagnosed as having, or being at risk of having) a lysosomal storage disease; the method comprising administering to the subject an effective amount of a compound of formula (I) as defined in claim 1.

37. The method of claim 36, wherein the method results in a reduction in GL-1 concentration in CSF of at least 30% (e.g. at least 40%, at least 50%, at least 60%, or at least 70%); and/or wherein the method results in the reduction in GL-1 concentration in the CSF is within 3 months of commencing treatment (e.g. within 2 months, 1 month, 3 weeks, 2 weeks, or 1 week of commencing treatment).

38. (canceled)

39. (canceled)

40. The method of claim 1, wherein said compound, or pharmaceutically acceptable salt or prodrug thereof, is administered orally; and/or wherein said subject is administered a daily dose of about 2 mg to about 30 mg of said compound, or pharmaceutically acceptable salt or prodrug thereof (e.g. from 2 mg to 20 mg, or from 2 mg to 10 mg, or from 4 mg to 8 mg, or from 10 to 20 mg, from 4 mg to 15 mg, or a dose selected from 4, 8, and 15 mg).

41. (canceled)

42. (canceled)

43. The method of claim 1, wherein the method prevents, reduces, or reverses deterioration in attention and concentration, executive functions, memory (e.g. working memory), language, visuo-constructional skills, conceptual thinking, calculations, orientation, decision making, and/or problem solving.

44. The method of claim 1, wherein the method is effective to improve cognitive ability or reduce cognitive deficits in the subject as measured by a reduction in the time taken to complete the trail-making test (TMT), TMT-A and/or TMT-B, a reduction in the difference between TMT-A time and TMT-B time (TMT-B−TMT-A), for example, a reduction of at least 10%, or at least 20%, or at least 30%, or at least 40%, or at least 50% (e.g. wherein TMT-A decreases by 5-20%, and/or TMT-B decreases by 25-30%, and/or [TMT-B−TMT-A] decreases by 25-30%).

45. (canceled)

46. The method of claim 1, wherein the subject is a heterozygous carrier of one or more glucocerebrosidase 1 gene (GBA1) mutations, and the compound is a malate acid addition salt of (S)-quinuclidin-3-yl (2-(2-(4-fluorophenyl)thiazol-4-yl)propan-2-yl)carbamate and is orally administered at a daily dose of about 15 mg (measured as the equivalent amount of free base).

47. The method of claim 46, wherein the concentration of the compound (measured as the equivalent amount of free base) in the CSF of the subject is at least 4 ng/ml (e.g. at least 8 ng/ml, or at least 10 ng/ml), within 3 months of commencing treatment (e.g. within 2 months, 1 month, 3 weeks, 2 weeks, or 1 week of commencing treatment).

48. The method of claim 46, wherein the method results in increased blood flow in the brain (e.g. in one or more of the frontal, occipital, parietal, or temporal lobes), for example, as shown by fMRI imaging and/or increased nodal connectivity in the brain (e.g. between posterior and anterior aspects of the brain, and/or between occipital-parietal structures and frontal, temporal, and/or limbic structures, for example, as shown by fMRI imaging).

49-56. (canceled)
